(19) United States
(12) Patent Application Publication
(10) Pub. No.: US 2022/0175850 A1
(43) Pub. Date: Jun. 9, 2022

(54) COMPOSITIONS AND METHODS FOR CANCER THERAPY

(71) Applicant: Houn Simon Hsia, Irvine, CA (US)

(72) Inventor: Houn Simon Hsia, Irvine, CA (US)

(21) Appl. No.: 17/599,981

(22) PCT Filed: Mar. 31, 2020
(86) PCT No.: PCT/US2020/026000

§ 371 Date: Sep. 29, 2021

Related U.S. Application Data
(60) Provisional application No. 62/827,429, filed on Apr. 1, 2019.
(60) Provisional application No. 62/895,421, filed on Sep. 3, 2019.

(51) Int. Cl. A61K 35/60; A61K 33/04; A61K 35/14; A61P 35/00

(52) U.S. Cl. CPC A61K 35/60; A61K 33/04; A61N 5/10; A61P 35/00; A61K 35/14

(57) ABSTRACT

Compositions and methods are described in which a nutritional supplement (such as a supplement that includes chromium and certain plant-derived materials and/or a supplement that includes a selenium yeast peptide complex and fish oil is used to provide an immunotherapeutic effect, which can be demonstrated by modulation of cell-surface markers targeted by conventional immunotherapeutic drugs. The nutritional supplements can be used in combination with other antineoplastic therapies, such as radiation and/or chemotherapeutic agents. Such combination therapy can provide a synergistic effect in regard to modulation of cell surface markers that serve as immunotherapy targets.

This application claims the benefit of U.S. Provisional Patent Application No. 62/895,421 filed on Sep. 3, 2019 and U.S. Provisional Patent Application No. 62/827,429 filed on Apr. 1, 2019. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is nutritional supplements, particularly application of nutritional supplements to immunotherapies used in the treatment of cancer.

### BACKGROUND

The background description includes information that may be useful in understanding the present invention. It is not an admission that any of the information provided herein is prior art or relevant to the presently claimed invention, or that any publication specifically or implicitly referenced is prior art.

Immunotherapeutic approaches to treating cancer exploit elements of the patient's own immune system to treat the disease. Such immunotherapies can involve the administration of antibodies, cytokines, and/or cells retrieved from the patient and treated prior to re-administration.

Antibodies utilized in immunotherapy are typically directed to cell surface markers expressed by tumor cells, in order to activate the body's complement system or otherwise identify the cells to the immune system. Alternatively, such antibodies can be directed to cell surface receptors and interfere with down regulation of T-cell activity by cancer cells. Antibodies used for this purpose include Alemtuzumab (a monoclonal antibody directed to CD52 and which activates complement), Atezomlizumab (a monoclonal antibody directed to PD-L1 and which interferes with T-cell deactivation), and Ilipimumab (a monoclonal antibody directed to CTLA4, shifting the T-cell balance towards cytotoxicity). Unfortunately use of these therapeutic antibodies is associated with unwanted side effects, including precipitation of autoimmune disease, increased rate of infections, and neurological disorders.

Immunotherapy utilizing cytokines is directed to provoking an immune response to the tumor cells, which can themselves produce cytokines that reduce immune response. Cytokines used for immunotherapy include IFNα (used in treatment of hairy-cell leukemia, AIDS-related Kaposi's sarcoma, follicular lymphoma, chronic myeloid leukemia, and melanoma), IFNβ, and interleukin 2 (used in treatment of malignant melanoma and renal cell carcinoma). While these cytokines are known to have a variety of effects on the immune system the exact mechanism by which they attack cancer is not clear. Unfortunately, administration of these cytokines is associated with flu-like symptoms.

Immunotherapies utilizing cells involve removal of cells from the patient, activation and expansion of the cells in culture, and return of the activated cells to the patient. For example, Provenge is used to treat prostate cancer, and involves the removal of antigen presenting dendritic cells from the blood by leukapheresis, incubating them with a fusion protein made from elements of GM-CSF and a prostatic acid phosphatase, and reinfusing. The resulting improved presentation of cancer-specific antigens to the immune system is intended to improve the immune response. In another approach, CAR-T immunotherapy removes T cells and genetically modifies them to express a chimeric receptor that specifically recognizes target cancer cells. These modified T cells are returned to the patient, where it is hoped that they selectively target the cancer cells. Unfortunately, such approaches are expensive and time consuming, can cause flu-like symptoms, and have produced mixed results.

Thus, there is still a need for a simple and well tolerated immunotherapeutic approach to treating cancer.

### SUMMARY OF THE INVENTION

The inventive subject matter provides compositions and methods in which a nutritional supplement is provided that modulates the expression of immune checkpoint proteins, immunotherapy targets, proteins associated with angiogenesis, and/or proteins associated with metastasis in cancer cells, which can be resistant to chemotherapy. Such cancer cells can be present in tissue culture or as a tumor. Such a nutritional supplement can be used in combination with chemotherapeutic drugs and/or radiotherapy.

One embodiment of the inventive concept is a method for providing immunotherapy to an individual with cancer (which can be drug resistant) by administering a nutritional supplement that includes selenium and fish oil (e.g. as shown in Table 1) in an amount sufficient to modify expression of a biological marker associated with an anti-neoplastic immune function. The nutritional supplement is provided in the absence of chemotherapy and/or in the absence of radiotherapy. In some embodiments the nutritional supplement is provided in combination with radiotherapy, thereby providing a synergistic effect in modifying expression of the biological marker. Suitable biological markers include AXL, HSP90, p-mTOR, PDL-1, EGFR, HDAC1, p-H2X, p-Akt, pSmad, mTOR, p-PTEN, p-STAT3, CXCR4, and STAT3. In some embodiments expression is increased for PD-1, CTLA4, FOXP3, CD8, PTEN, and/or p-P53. In some embodiments the ratio of expression of CD4 to expression of CD8 is reduced. In some embodiments the percentage of CD3+ T cells, CD3+CD4+ T cells, or CD4+CD8+ T cells in the individual's spleen is increased. In some embodiments of the inventive concept the immunotherapy provided by the nutritional supplement reduces expression of a biological marker associated with a stem cell characteristic or metastatic potential in tumor cells, such as CD24, CD29, CD31, VEGF, and MMP-9.

Another embodiment of the inventive concept is a method of activating immune cells to enhance anti-tumor activity by isolating an immune cell from an individual to be treated for cancer, contacting the isolated immune cell with an activating formulation comprising selenium and fish oil in an amount effective to modulate expression of a protein associated with immune cell activation to generate an activated immune cell, and returning the activated immune cell to the individual. A suitable formulation and/or elements thereof is shown in Table 1. In some embodiments of the inventive concept the activated immune cell is clonally expanding to generate a population of activated immune cells that is returned to the individual. In some embodiments the immune cell is clonally expanded prior to contacting with the activating formulation. In some embodiments the immune cell and/or the activated immune cell is genetically modified prior to returning to the individual. In some embodiments the individual is irradiated prior to isolating the immune cell.

Another embodiment of the inventive concept is a method of modulating expression of an immune checkpoint or immunotherapy-associated protein in a cell, by administering a supplement comprising selenium and fish oil (such as the supplement shown in Table 1). The supplement can be administered to provide a concentration of at least 200 ng/mL of selenium. The supplement can be administered to provide a concentration of at least 75 μM fish oil. Suitable fish oils include a fish oil with DHA and EPA in an about 2:3 weight ratio. The cell can be a cancer cell, which can have stem cell characteristics and/or be resistant to a chemotherapeutic drug. In some embodiments the method includes administering a chemotherapeutic drug and/or administering radiotherapy to the cell. In such embodiments the supplement can be administered prior to initiation of radiotherapy.

The modulation can be a reduction in expression, for example when the immune checkpoint or immunotherapy-associated protein is PD-L1, p-HSP27, vimentin, p-mTOR, p-p38, β-catenin, ABCG2, CD133, N-cadherin, p-MET, COX-2, GRP78, CD24, CD29, EGFR, HDAC1, p-H2X, p-Akt, MMP-9, CTLA4, CD28, CD86, C31, and/or STAT3. The modulation can be an increase in expression, and wherein the immune checkpoint or immunotherapy-associated protein is selected from the group consisting of PD-1, p-AMPKα, E-cadherin, CHOP, FOXP3, Nkp46, CD8, IL2, and/or PTEN.

Another embodiment of the inventive concept is a method for reducing circulating tumor cells in an animal with cancer by administering a nutritional supplement comprising selenium and fish oil (such as the supplement shown in Table 1) to the animal and administering chemotherapy to the animal. The supplement can be administered to provide a concentration of at least 200 ng/mL of selenium. The supplement can be administered to provide a concentration of at least 75 μM fish oil, which preferably has a DHA to EPA weight ratio of about 2:3.

Another embodiment of the inventive concept is the use of a supplement that includes selenium and fish oil to modulate expression of an immune checkpoint or immunotherapy-associated protein in a cell. The supplement can provide a concentration of at least 200 ng/mL of selenium to the cell following administration. The supplement can provide a concentration of at least 75 μM fish oil to the cell following administration. The fish oil preferably includes DHA and EPA in an about 2:3 weight ratio. The cell can be a cancer cell, such as a cancer cell that has stem cell characteristics and/or a cancer cell that is resistant to a chemotherapeutic drug. In some embodiments the supplement is used in combination with a chemotherapeutic drug and/or in combination with radiotherapy. In such an embodiment the supplement can used prior to initiation of radiotherapy.

The modulation can be a reduction in expression, such as when the immune checkpoint or immunotherapy-associated protein is PD-L1, p-HSP27, vimentin, p-mTOR, p-p38, □-catenin, ABCG2, CD133, N-cadherin, p-MET, COX-2, GRP78, CD24, CD29, EGFR, HDAC1, p-H2X, p-Akt, MMP-9, CTLA4, CD28, CD86, C31, and/or STAT3. The modulation can be an increase in expression, such as when the immune checkpoint or immunotherapy-associated protein is PD-1, p-AMPKα, E-cadherin, CHOP, FOXP3, Nkp46, CD8, and/or PTEN.

Another embodiment of the inventive concept is the use of a nutritional supplement that includes selenium and fish oil in combination with chemotherapy for reducing circulating tumor cells in an animal with cancer. The nutritional supplement provides a concentration of at least 200 ng/mL of selenium following administration. The nutritional supplement can provide a concentration of at least 75 μM fish oil following administration. In preferred embodiments the fish oil includes DHA and EPA in an about 2:3 weight ratio.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1: Mechanism and markers of Iressa resistance.

FIG. 2: Reduction of Axl expression in Iressa-resistant lung cancer cells by 24 hours of treatment with a nutritional supplement that includes selenium and fish oil.

FIG. 3: Regulation of Axl expression and processing.

FIG. 4: Axl and HSP90 expression in Iressa-sensitive and Iressa-resistant lung cancer cell lines.

FIG. 5A to 5E: FIG. 5A: Reduction of Axl and HSP90 expression in Iressa-resistant lung cancer cells by 72 hours of treatment with a nutritional supplement that includes selenium and fish oil. While Iressa alone has no apparent effect, a synergistic effect is apparent on combined treatment. FIG. 5B: Negative modulation of heat shock protein expression induced in A549 human lung cancer sphere cells by use of selenium and fish oil in combination. FIG. 5C: Overexpression of ABCG2, CD133, and CD44 in cells resistant to a chemotherapeutic drug. FIG. 5D: Modulation of p-p38, p-HP27, -catenin, ABCG2, CD1333, N-cadherin, and E-cadherin expression induced in chemotherapy resistant human lung cancer cells by use of selenium and fish oil in combination, in the presence and absence of 1 μM Iressa. FIG. 5E: Modulation of p-MET, -catenin, COX-2, GRP78, p-p38, p-AMPK?, and CHOP expression induced in chemotherapy resistant human lung cancer cells by use of selenium and fish oil in combination, in the presence and absence of 0.1 μM Iressa.

FIGS. 6A and 6B: FIG. 6A: Effects of a nutritional supplement that includes selenium and fish oil on phosphorylated mTOR (p-mTOR) in drug resistant HCC827GR cells after 72 hours. FIG. 6B: Negative modulation of PD-L1 expression induced in chemotherapy resistant human lung cancer cells by use of selenium and fish oil in combination.

FIGS. 7A to 7C: FIG. 7A: Reduction in PD-L1 expression in human lung cancer cells by treatment with a nutritional supplement containing selenium and fish oil for 72 hours. FIG. 7B: Effects of 72 hours exposure to fish oil and selenium on PD-L1 expression in A549 sphere cells. FIG. 7C: Effects of selenium and fish oil of PDL-1 and PD-1 expression in stem-cell like sphere cells following treatment with selenium and fish oil (with and without co-treatment with a chemotherapy drug).

FIGS. 8A and 8B: FIG. 8A: Modulation of PD-L1 expression in an animal tumor model for triple negative breast cancer using a nutritional supplement that includes selenium and fish oil. Synergistic effects when used in combination with chemotherapeutic drugs are apparent.

FIG. 8B: Modulation of PD-L1 and PD-1 expression in primary (tumor) and metastatic (breast) sites an animal tumor model for triple negative breast cancer using a nutritional supplement that includes selenium and fish oil. Synergistic effects when used in combination with chemotherapeutic drugs are apparent.

FIGS. 9A to 9J: FIG. 9A: A typical protocol for evaluation of modulation of immunotherapy target proteins with cotherapy using a chemotherapeutic agent in an animal model using different amounts of selenium and fish oil supplementation. FIG. 9B: In vivo studies of the effect of different doses of nutritional supplement that includes selenium and fish oil on PD-1, PD-L1, CTLA4, and FOXP3 expression in primary breast cancer tissue. FIG. 9C: Modulation of tumor CD24 expression using different amounts of selenium and fish oil in combination with Avastin or Taxol in an animal tumor model. FIG. 9D: Modulation of tumor CD29 expression using different levels of selenium and fish oil in combination with Avastin or Taxol in an animal tumor model. FIG. 9E: Modulation of tumor EGFR expression using different levels of selenium and fish oil in combination with Avastin or Taxol in an animal tumor model. FIG. 9F: Modulation of tumor p-mTOR expression using different levels of selenium and fish oil in combination with Avastin or Taxol in an animal tumor model. FIG. 9G: Modulation of tumor HDAC1 and p-H2X expression using different levels of selenium and fish oil in combination with Taxol in an animal tumor model. FIG. 9H: Modulation of tumor p-Akt expression using different levels of selenium and fish oil in combination with Avastin or Taxol in an animal tumor model. FIG. 9I: Negative modulation of vimentin expression induced in A549 human lung cancer sphere cells by use of selenium and fish oil in combination. FIG. 9J: Positive modulation of p-AMPKα expression and negative modulation of p-mTOR expression induced in A549 human lung cancer sphere cells by use of selenium and fish oil in combination.

FIGS. 10A to 10E: FIG. 10A: Study design for antibody-based anti-tumor immunotherapy in combination with a nutritional supplement containing fish oil and selenium. FIG. 10B: Effects of H2 anti-PD-1 antibody, a nutritional supplement that includes fish oil and selenium, and the antibody and supplement in combination on the percentage of CD3+ T cells found in the spleens of tumor-bearing mice. FIG. 10C: Effects of H2 anti-PD-1 antibody, a nutritional supplement that includes fish oil and selenium, and the antibody and supplement in combination on the percentage of CD3+/CD4+ T cells found in the spleens of tumor-bearing mice. FIG. 10D: Effects of H2 anti-PD-1 antibody, a nutritional supplement that includes fish oil and selenium, and the antibody and supplement in combination on the percentage of CD3+/CD4+ T cells found in the spleens of tumor-bearing mice. FIG. 10E: Effects of H2 anti-PD-1 antibody, a nutritional supplement that includes fish oil and selenium, and the antibody and supplement in combination on the percentage of dendritic cells found in the spleens of tumor-bearing mice.

FIG. 11: A typical dosing schedule for nutritional supplements of the inventive concept in combination with taxol or avastin.

FIG. 12: Plasma selenium concentrations in tumor bearing mice co-treated with nutritional supplement formulation containing different amounts of selenium and different chemotherapeutic agents.

FIG. 13: Selenium concentrations in tumor tissue obtained from tumor bearing mice co-treated with nutritional supplement formulation containing different amounts of selenium and different chemotherapeutic agents.

FIG. 14: Reduction in EGFR expression in tumors of tumor-bearing animals treated with a chemotherapeutic agent and a nutritional supplement containing fish oil and different amounts of selenium.

FIG. 15: Reduction in p-mTOR in tumors of tumor-bearing animals treated with a chemotherapeutic agent and a nutritional supplement containing fish oil and different amounts of selenium.

FIG. 16: Reduction in histone deacetylase 1 (HDAC1 and p-H2X in tumors of tumor-bearing animals treated with a chemotherapeutic agent and a nutritional supplement containing fish oil and different amounts of selenium.

FIG. 17: Reduction in p-Akt phosphorylated at Ser473 or at Thr308 in tumors of tumor-bearing animals treated with a chemotherapeutic agent and a nutritional supplement containing fish oil and different amounts of selenium.

FIG. 18: Reduction in p-Smad in the nuclei of tumors of tumor-bearing animals treated with a chemotherapeutic agent and a nutritional supplement containing fish oil and different amounts of selenium.

FIGS. 19A and 19B: FIG. 19A: In vivo studies of the effect of a nutritional supplement that includes selenium and fish oil on CD24 and CD29 expression in breast cancer tissue. FIG. 19B: In vivo studies of the effect of different doses of a nutritional supplement that includes selenium and fish oil on CD 24 and CD29 expression in breast cancer tissue.

FIG. 20: In vivo studies of the effect of different doses of a nutritional supplement that includes selenium and fish oil on VEGF, CD24, CD29, and MMP-9 expression in a metastatic brain tumor site.

FIGS. 21A and 21B: FIG. 21A: Relative CD31 expression levels of metastatic tissue in mice implanted with human lung cancer cells. C=control (unimplanted), T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation. FIG. 21B: Relative CD31 expression levels of primary tumor site tissue in mice implanted with human lung cancer cells. C=control (unimplanted), T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation.

FIGS. 22A and 22B: FIG. 22A: Relative CD8 expression levels of metastatic tissue in mice implanted with human lung cancer cells. C=control (unimplanted), T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation. FIG. 22B: Relative CD8 expression levels of primary tumor site tissue in mice implanted with human lung cancer cells. T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation.

FIGS. 23A and 23B: FIG. 23A: Relative CD4/CD8 expression levels of metastatic tissue in mice implanted with human lung cancer cells. C=control (unimplanted), T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation. FIG. 23B: Relative CD4/CD8 expression levels of primary tumor tissue in mice implanted with human lung cancer cells. T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation.

FIGS. 24A and 24B: FIG. 24A: CTLA4 expression levels of metastatic tissue in mice implanted with human lung cancer cells. C=control (unimplanted), T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation. FIG. 24B: CTLA4 expression levels of primary tumor tissue in mice implanted with human lung cancer cells. T=tumor cells implanted with no treatment, PTN=tumor implanted animals treated with a nutritional supplement including selenium and fish oil prior to implantation, TN=tumor implanted animals treated with a nutritional supplement including selenium and fish oil at the time of implantation, TR=tumor implanted animals treated with radiation, PTRN=tumor implanted animals treated with a nutritional supplement including selenium and fish oil prior to implantation and treated with radiation, TRN=tumor implanted animals treated with a nutritional supplement including selenium and fish oil at the time of implantation and with radiation.

FIGS. 25A and 25B: FIG. 25A: PD-1 and PD-L1 expression in tumors of animal models of breast cancer. Animals were treated with a nutritional supplement containing selenium and fish oil (-N), taxol (-tax), adriamycin (-adyri), avastin, or a combination of chemotherapeutic agent and nutritional supplement. Histograms are normalized relative to expression in untreated tumors. FIG. 25B: Ratio of PD-1 to PD-L1 expression in tumors of animal models of breast cancer. Animals were treated with a nutritional supplement containing selenium and fish oil (-N), taxol (-tax), adriamycin (-adyri), avastin, or a combination of chemotherapeutic agent and nutritional supplement.

FIG. 26: Human clinical trial of a supplement containing selenium and fish oil. Subjects receiving low doses of the supplement (G1) are shown on the left, medium doses of the supplement (G2) in the center, and high doses of the supplement (G3) on the right. Results are expressed as the change in white blood cell PD-1 content relative to tumor cell PDL-1 content between week 1 and week 16 of the study.

FIG. 27: Effect of cotherapy with chemotherapeutic agents and a nutritional supplement containing selenium and fish oil provided at low (-S), mid (-M), and high (-H) doses. Results from tumors from animal subjects treated with taxol are shown on the left; results obtained from tumor samples from animals treated with avastin are shown on the right.

FIG. 28: Effect of cotherapy with chemotherapeutic agents and a nutritional supplement containing selenium and fish oil provided at low (-S), mid (-M), and high (-H) doses. Results from tumors from animal subjects treated with taxol are shown on the left; results obtained from tumor samples obtained from animals treated with adriamycin are shown on the right. Quantitation relative to the untreated control is shown below each band.

FIG. 29: Effect of cotherapy with chemotherapeutic agents and a nutritional supplement containing selenium and fish oil provided at low (-S), mid (-M), and high (-H) doses on tumor CXCR4 expression. Results from tumors from animal subjects treated with avastin and avastin in combination with a nutritional supplement containing selenium and fish oil are shown. Quantitation relative to the untreated control is shown below each band.

FIG. 30: Typical study design for cotherapy with radiation and a nutritional supplement containing selenium and fish oil.

FIGS. 31A and 31B: FIG. 31A: Effects of radiation therapy and treatment with a nutritional supplement containing selenium and fish oil on CD8 expression in lung tissue in an animal model of lung cancer. C=untreated control, T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12. FIG. 31B: Effects of radiation therapy and treatment with a nutritional supplement containing selenium and fish oil on CD8 expression in primary tumor implantation sites in an animal model of lung cancer. T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12.

FIGS. 32A and 32B: FIG. 32A: Effects of radiation therapy and treatment with a nutritional supplement containing selenium and fish oil on the CD4/CD8 ratio of lung tissue in an animal model of lung cancer. C=untreated control, T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12. FIG. 32B: Effects of radiation therapy and treatment with a nutritional supplement containing selenium and fish oil on the CD4/CD8 ratio in primary tumor implantation sites in an animal model of lung cancer. T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12.

FIG. 33: Effects of radiation therapy and treatment with a nutritional supplement containing selenium and fish oil on STAT3 expression in primary tumor implantation sites in an animal model of lung cancer. T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12.

FIG. 34: Effects of radiation therapy and treatment with a nutritional supplement containing selenium and fish oil on PTEN expression in primary tumor implantation sites in an animal model of lung cancer. T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12.

FIGS. 35A and 35B: FIG. 35A: Effects of radiation therapy and treatment with a nutritional supplement containing selenium and fish oil on CTLA-4 expression in primary tumor implantation sites in an animal model of lung cancer. T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12. FIG. 35B: Effects of radiation therapy and treatment with a nutritional supplement containing selenium and fish oil on the CTLA-4 expression of lung tissue in an animal model of lung cancer. C=untreated control, T=tumor cells implanted, no treatment, PTN=tumor cells implanted, treatment with nutritional supplement started immediately, TN=tumor cells implanted, treatment with nutritional supplement started on day 8, TR=tumor cells implanted, radiotherapy on days 8, 10, and 12, PTRN=tumor cells implanted, treatment with nutritional supplement started on day 8, TR=tumor cells implanted and treatment with nutritional supplement started immediately, radiotherapy on days 8, 10, and 12, TRN=tumor cells implanted, treatment with nutritional supplement started on day 8, radiotherapy on days 8, 10, and 12.

FIG. 36: Results of studies of dose dependent modulation of immune checkpoint, angiogenesis, and invasiveness associated protein in tumors of animal models of triple negative breast cancer using selenium and fish oil in combination with either Taxol or Adriamycin.

FIG. 37: Results of studies of dose dependent modulation of immune checkpoint, angiogenesis, and invasiveness associated protein in tumors of animal models of triple negative breast cancer using selenium and fish oil in combination with either Taxol or Adriamycin.

FIG. 38: Results of studies of dose dependent modulation of Nkp46 in tumors of animal models of triple negative breast cancer using selenium and fish oil in combination with either Taxol or Adriamycin.

FIG. 39: Results of studies of dose dependent modulation of CD28, CD86, and CD80 in tumors of animal models of triple negative breast cancer using selenium and fish oil in combination with either Taxol or Avastin.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

The inventive subject matter provides compositions and methods in which a nutritional supplement (such as a supplement that includes chromium and certain plant-derived materials (NutraWell) and/or a supplement that includes a selenium yeast peptide complex and fish oil) is used to provide an immunotherapeutic effect, which can be demonstrated by modulation of cell-surface markers targeted by conventional immunotherapeutic drugs. In some embodiments such nutritional supplements are provided in combination with other antineoplastic therapies, such as radiation and/or chemotherapeutic agents. In some embodiments such combination therapy can surprisingly provides a significant synergistic effect in regard to modulation of cell surface markers that serve as immunotherapy targets.

One should appreciate that the disclosed techniques provide many advantageous technical effects including providing novel and well tolerated approach to modulating expression of cell surface markers associated with immunotherapy, but also in enhancing or complementing the effectiveness of current antineoplastic protocols.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

While the some findings described below are directed to the use of fish oil and a selenium source, the Applicant notes that the nutritional supplement formulation provided in Table 1 incorporates fish oil and selenium yeast components (such as peptides and/or amino acids prepared from selenium yeast). As such effects found in fish oil and selenium yeast studies can be extended to the use of this nutritional supplement or to formulations that include fish oil and selenium yeast in combination with one or more of the remaining components of the formulation shown in Table 1. The supplement formulation shown in Table 1 is a minimal dose formulation that has been found to have a high level of acceptance and to have unanticipated beneficial effect in regulating the expression of cell surface markers associated with antineoplastic immunotherapies. As shown below, such a nutritional supplement can also complement and/or enhance the effects of conventional antineoplastic therapies when used in combination. The formulation for a typical nutritional supplement of the inventive concept is shown below in Table 1.

TABLE 1

| | Minimum | Maximum | Unit |
|---|---|---|---|
| Component | | | |
| Maltodextrin | 10000 | 50000 | mg |
| Whey Protein Isolate | 5000 | 60000 | mg |
| Whey Protein Concentrate | 1000 | 50000 | mg |
| Fructooligosaccharides/Inulin | 40 | 15000 | mg |
| Granulated Honey | 1000 | 9000 | mg |
| Oat Fiber | 500 | 15000 | mg |
| Natural French Vanilla Flavor | 500 | 20000 | mg |
| Soy Protein | 500 | 50000 | mg |
| Brownulated Powdered Brown Sugar | 500 | 10000 | mg |
| Natural Vanilla Masking Flavor | 500 | 5000 | mg |
| Lecithin | 200 | 10000 | mg |
| Milk, Non-fat | 50 | 5000 | mg |
| Rice Protein Powder | 50 | 5000 | mg |
| Calcium Caseinate | 50 | 2000 | mg |
| Oils | | | |
| Flax Seed Oil | 100 | 7000 | mg |
| Canola Oil | 100 | 7000 | mg |
| Borage Oil | 100 | 7000 | mg |
| Olive Oil | 100 | 7000 | mg |
| Fish Oil | 150 | 5000 | mg |
| Pure Lemon Oil | 100 | 1000 | mg |
| Pure Orange Oil | 50 | 1000 | mg |
| Mixed Tocopherols | 0.5 | 200 | mg |
| Vitamins/Minerals | | | |
| Potassium Phosphate | 200 | 1500 | mg |
| Calcium | 100 | 7000 | mg |
| Choline Bitartrate | 150 | 2500 | mg |
| Sodium | 100 | 2000 | mg |
| Ascorbic Acid | 50 | 3000 | mg |
| Potassium | 50 | 2000 | mg |
| Magnesium | 50 | 600 | mg |
| Selenium | 30 | 4000 | mcg |
| Chromium | 30 | 3000 | mcg |
| Molybdenum | 30 | 2000 | mcg |
| Inositol | 10 | 5000 | mg |
| Zinc | 5 | 200 | mg |
| Dry Vitamin E Acetate | 5 | 2000 | IU |
| Niacinamide | 5 | 500 | mg |
| Iron | 3 | 100 | mg |
| Calcium Pantothenate | 3 | 200 | mg |
| Manganese | 3 | 300 | mg |
| Beta Carotene | 1 | 500 | mg |
| Copper Gluconate | 1 | 50 | mg |
| Vitamin D3 | 25 | 10,000 | IU |
| Vitamin K2 | 2 | 1000 | mcg |
| Pyridoxine HCl (Vitamin B6) | 0.5 | 300 | mg |
| Potassium Iodide | 0.5 | 1500 | mg |
| Riboflavin (Vitamin B2) | 0.5 | 1000 | mg |
| Thiamine Hydrochloride (Vitamin B1) | 0.5 | 2500 | mg |
| Vitamin K1 | 1 | 500 | mcg |
| Vitamin A | 500 | 100,000 | IU |
| Folic Acid | 100 | 10000 | mcg |
| d-Biotin | 10 | 10000 | mcg |
| Vitamin B12 | 1 | 3000 | mcg |
| Amino Acids | | | |
| L-Carnitine | 300 | 30000 | mg |
| L-Glutamine | 500 | 60000 | mg |
| L-Arginine | 500 | 30000 | mg |
| Taurine | 50 | 2000 | mg |
| L-Lysine | 50 | 2000 | mg |
| Glycine | 5 | 1000 | mg |
| Proline | 5 | 1000 | mg |
| Antioxidants | | | |
| Alpha Lipoic Acid | 10 | 1000 | mg |
| Resveratrol | 15 | 1500 | mg |
| Co-Enzyme Q10 | 10 | 5000 | mg |
| Bacterial Cultures | | | |
| Lact. Acidophilus (app. 10 billion total) | 2 | 500 | mg |
| Bifido Bifidium (app. 10 billion total) | 2 | 500 | mg |
| Lac. Bulgaricus (app. 10 billion total) | 2 | 500 | mg |
| Bifido Longum (app. 10 billion total) | 2 | 500 | mg |
| Strep. Thermophilus (app. 10 billion total) | 2 | 500 | mg |
| Enzymes | | | |
| Papain | 5 | 100 | mg |
| Pepsin | 5 | 100 | mg |
| Lipase | 5 | 100 | mg |
| Bromelain | 5 | 100 | mg |
| Pancreatin | 0.5 | 100 | mg |
| Lactase | 1 | 100 | mg |
| Betaine HCl | 3 | 100 | mg |
| Plant Products | | | |
| Pineapple Juice Powder | 2 | 500 | mg |
| Papaya Fruit Powder | 2 | 500 | mg |
| Quercetin | 30 | 3000 | mg |
| EGCG | 25 | 600 | mg |
| OPC | 15 | 500 | mg |
| Anthocyanins | 15 | 5000 | mg |
| Ellagic Acid | 10 | 300 | mg |
| Astaxanthin | 2 | 90 | mg |
| Fucoidan | 20 | 1500 | mg |
| Mushroom Preparation | | | |
| Cordyceps | 5 | 6000 | mg |
| Ganoderma Lucidum | 15 | 10000 | mg |
| Shiitake | 40 | 15000 | mg |
| Maitake | 30 | 15000 | mg |
| Turkey Tail | 30 | 15000 | mg |

The composition shown in Table 1 includes components that have various physiological and biochemical effects, including anti-inflammatory activity, lowering of blood glucose levels, lowering of cholesterol, and anti-tumor activity. Other components provide supplementation of necessary vitamins, minerals, and amino acids at elevated levels. Other components (e.g. enzymes, lecithin) serve to aid in digestion and absorption of components of the composition when consumed. The combination of these complementary activities provides a synergistic effect that exceeds the simple additive effect of individual components. It should be appreciated that the composition shown in Table 1 also includes certain flavorants (e.g. brown sugar, honey, vanilla flavor and masking agent) that serve to improve palatability and acceptance. Certain components (e.g. honey, brown sugar, milk, rice protein, casein) can provide both flavor and caloric energy. The Inventor has found that the combination of flavorants described above is effective in providing compliance with consumption of the nutritional supplement in effective amounts. In some embodiments, such flavorants can be excluded without negatively impacting the effectiveness of the nutritional supplement.

G to It should be appreciated that Table 1 provides a description of a formulation providing a minimum daily dosage of the cited ingredients, and that the range expressed for each of these is considered to be disclosure of intermediate ranges and/or subranges within the cited range. For example, a range of 30 μg to 4,000 μg of selenium is indicative of a minimal formulation that provides 30 μg to 4,000μ of selenium per day, and can be instructive of formulations that include various subranges that lie within this range (e.g. 500 μg to 2,000 μg, 2,000 μg to 4,000 μg. etc.). The composition shown in Table 1 can be represented by a single formulation or by two or more formulations that in sum provide the component ingredients.

It should also be appreciated that Table 1 teaches a minimal formulation (or Low dose), and that embodiments of the inventive concept can include formulations or use of formulations that include higher doses or ranges, for example a Mid dose where the composition includes 50% more of the component ingredients (i.e. 1.5× over what is shown in Table 1) or a High dose where the composition includes 100% more of the component ingredients (i.e. 2× over what is shown in Table 1). Accordingly, Mid and High dose formulations encompass formulations in which such multipliers are applied to the minimal formulation shown in Table 1. For example, a range of 30 μg to 4,000 μg of selenium in the minimal formulation can be modified by a 1.5× multiplier to 45 μg to 6,000 μg in a Mid dose formulation and to 60 μg to 8,000 μg in a High dose formulation modified by a 2× multiplier. Intermediate ranges within these Mid and High dose formulation ranges are considered disclosed as described above. Such Mid or High dose compositions can be represented by a single formulation or by two or more formulations that in sum provide the component ingredients.

In some embodiments of the inventive concept a component or ingredient listed in Table 1 can be provided as a portion of a larger molecule, salt, or complex. For example, metals such as selenium, chromium, and/or molybdenum can be provided as selenium yeast, chromium yeast, and molybdenum yeast (respectively), or as components of such yeast formulations.

In other embodiments of the inventive concept the nutritional supplement can include fish oil and a selenium-yeast preparation, or one or more selenium-containing peptides or amino acids derived from such yeast. Such a selenium peptide can include selenocysteine and/or selenomethionine.

In preferred embodiments fish oil utilized in compositions and methods of the inventive concept has a DHA to EPA weight ratio of about 2:3. Within the context of this application the term “about” in this context is inclusive of a ±10% or a ±20% deviation from the nominal value.

It should be appreciated that some embodiments of the inventive concept are compositions and methods of using a nutritional supplement containing selenium and fish oil to treat cancer, whereas other embodiments are compositions and methods that modulate the amounts of specific biological markers in living cells, whereas still other embodiments of the inventive concept are directed to the use of such a supplement in combination with one or more conventional anti-cancer therapies, in particular immunotherapy.

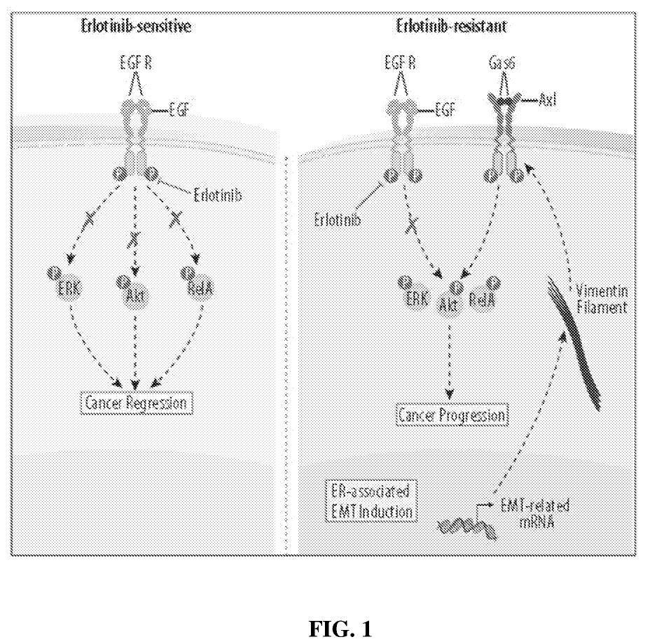

Some cancer cells are resistant to commonly used chemotherapeutic drugs, such as Iressa/Erlotinib. In some individuals such resistance occurs during the course of chemotherapeutic treatment, resulting in a loss of effectiveness and tumor progression. In others the cancer is resistant to the chemotherapeutic drug prior to treatment, allowing the tumor to progress as conventional chemotherapy is implemented. FIG. 1 illustrates a known mechanism for Iressa/Erlotinib resistance in tumor cells. Mutational activation of EGFR (a receptor tyrosine kinase or RTK) is common in non-small cell lung cancer, and leads to activation of ERK, Akt, and RelA that in turn promotes cancer progression. In non-resistant cells (left panel) Iressa/Erlotinib blocks the activity of EGF R, resulting in tumor regression. In resistant cells (right panel) Axl, another RTK, is overproduced and provides an alternate route for activation of ERK, Akt, and RelA that is not blocked by the drug. This Axl overexpression is linked to overexpression of vimentin, which suggests that endothelial mesenchymal transition may play a role in the development of Iressa/Erlotinib resistance.

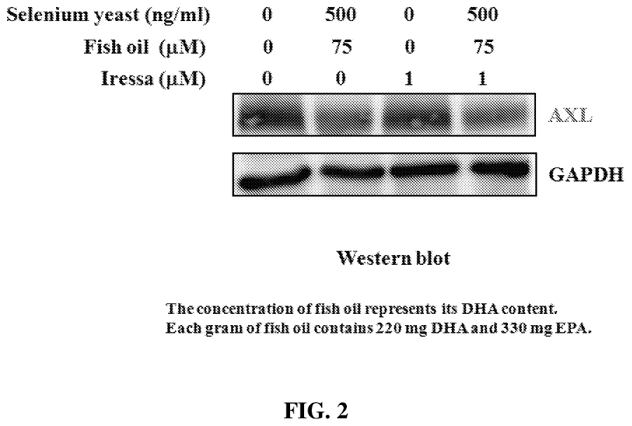

The HCC827GR cell line is a lung cancer cell line that is resistant to Iressa. As shown in FIG. 2 these cells produce large amounts of Axl, which is not reduced on treatment with Iressa. Surprisingly, however, application of selenium yeast and fish oil in combination dramatically reduces Axl expression in either the absence or presence of Iressa, thereby sensitizing these resistant cells to the drug. Nutritional supplements containing both selenium (for example, as selenium yeast) and fish oil can effectively reduce Axl expression in drug (for example, Iressa) resistant cells. Accordingly, patients having tumors whose drug resistance status is not known or is changing can be treated with either the nutritional supplement alone or a combination of the chemotherapy drug and the nutritional supplement.

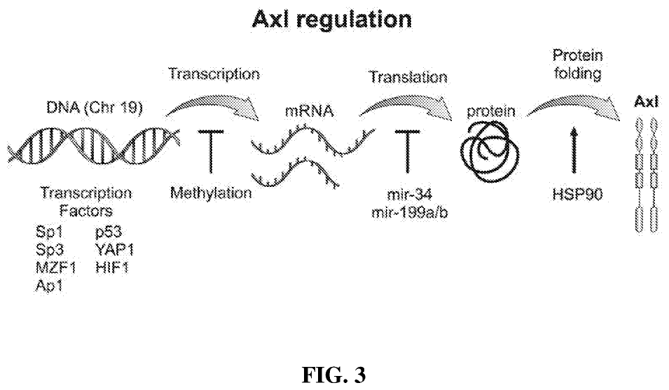

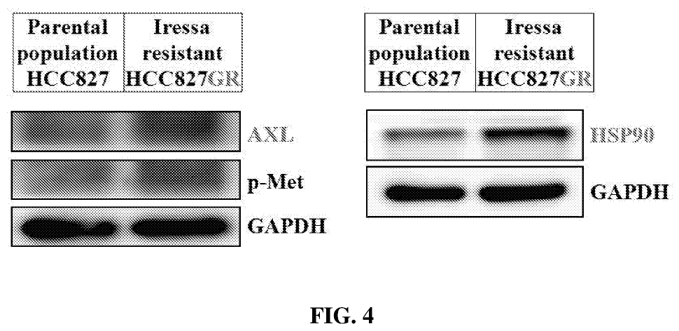

As shown in FIG. 3, proper folding of Axl is dependent on heat shock protein 90 (HSP90, a chaperonin). Since improper folding of Axl leads to increased rates of degradation, inhibition or reduced expression of HSP90 can result in decreased Axl. It is notable that both Axl and HSP90 expression is elevated in Iressa-resistant HCC827GR relative to the Iressa-sensitive parental HCC827 cell line, as shown in FIG. 4.

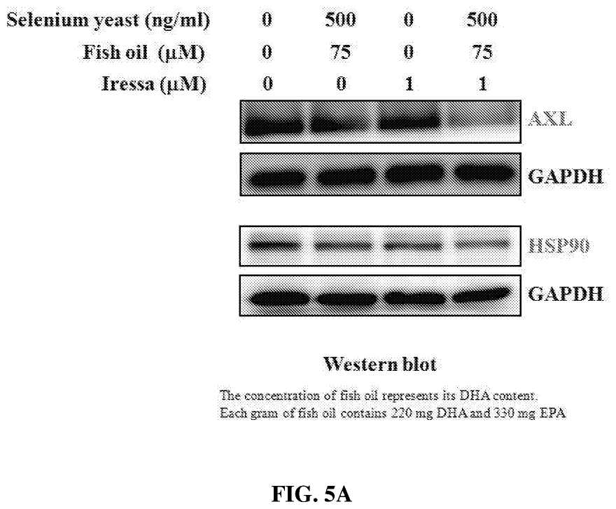

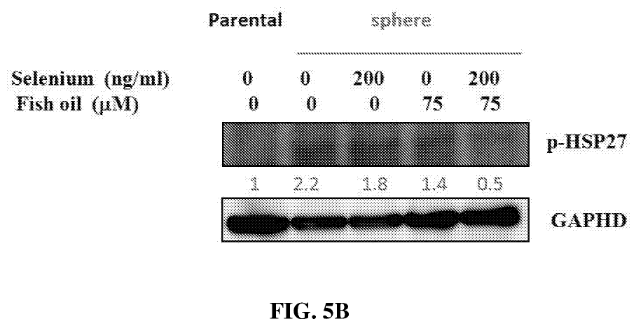

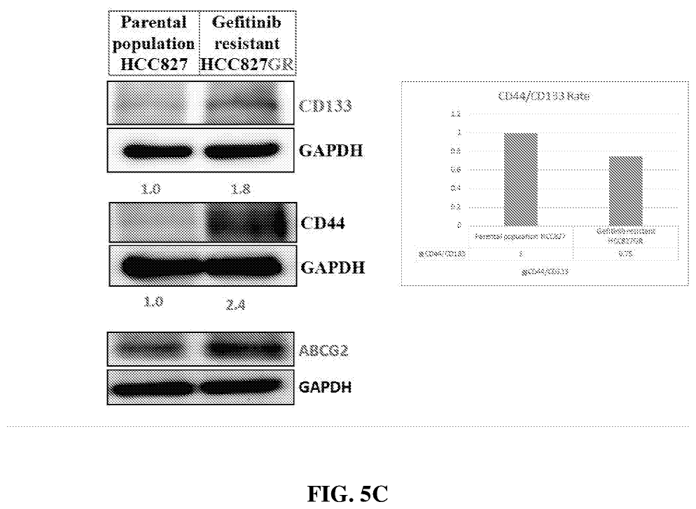

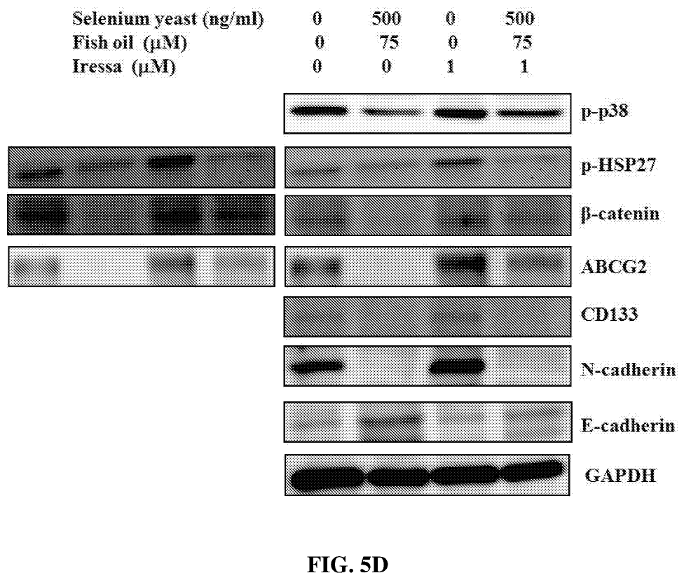

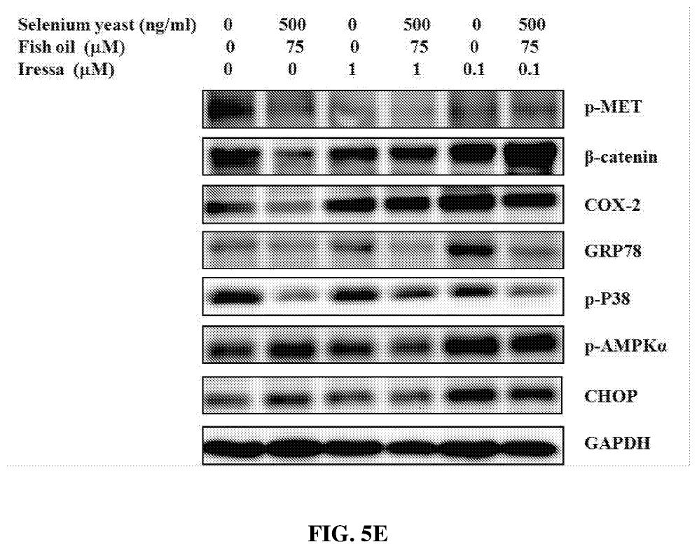

As shown in FIG. 5A, treatment of Iressa-resistant HCC827GR cells with a nutritional supplement that includes both selenium and fish oil for 72 hours reduces both Axl and HSP90 expression, whereas treatment with Iressa alone has no apparent effect. Remarkably, a considerable synergistic effect is apparent in the reduction of Axl expression when a combination of Iressa and the nutritional supplement is used.

The Inventor has found that treatment with fish oil and selenium (or with a nutritional supplement containing fish oil and selenium) can modulate expression of heat shock proteins other than HSP90. The Inventor has found that treatment with selenium and fish oil can modulate expression of heat shock proteins, for example p-HSP27. Such heat shock proteins are thought to provide a protective effect and are frequent targets of immunotherapy. As shown in FIG. 5B, treatment with a combination of fish oil and selenium was found to provide a synergistic effect in reducing p-HSP27 expression that was not found for selenium or fish oil alone.

Immunotherapy is a treatment modality that remains available when tumor cells are resistant to chemotherapeutic drugs. As shown in FIG. 5C, drug resistant human lung cancer cells (HCC827GR) show overexpression of immunotherapy targets such as CD133, CD44, and ABCG2 relative to the susceptible parental cell line. FIGS. 5D and 5E show the effects of treatment with fish oil and selenium on expression of various immunotherapy targets and other tumor markers (i.e. p-P38, p-HSP27, β-catenin, ABCG2, CD133, N-cadherin, E-cadherin, p-MET, COX-2, GRP78, p-AMPKα, and CHOP) in this resistant cell line (HCC827Gr) in the presence and absence of different concentrations of the chemotherapy drug Iressa. It should be appreciated that in some instances cotherapy with a chemotherapeutic drug appears to partially offset the effects of fish oil and selenium in combination, particularly at higher concentrations of the drug.

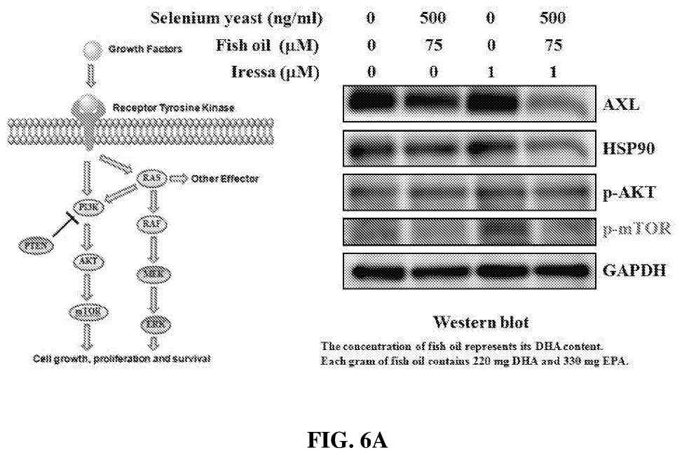

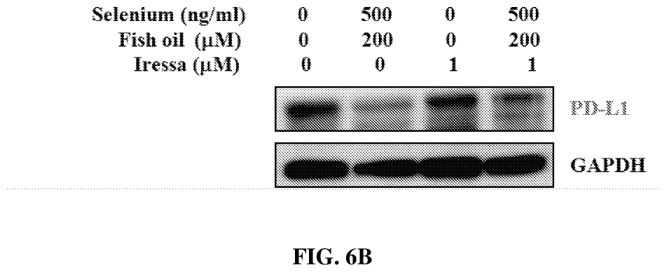

As shown in the left panel of FIG. 6A, activation of AKT by Receptor Tyrosine Kinase also results in increased phosphorylated mTOR, which in turn results in increases in tumor cell growth and proliferation in drug-resistant tumor cells. Inventors have found that use of a nutritional supplement that includes selenium and fish oil results in a reduction in Axl, HSP90, phosphorylated ATK, and phosphorylated mTOR (see right panel of FIG. 6).

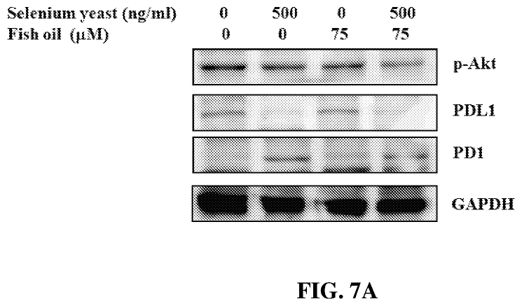

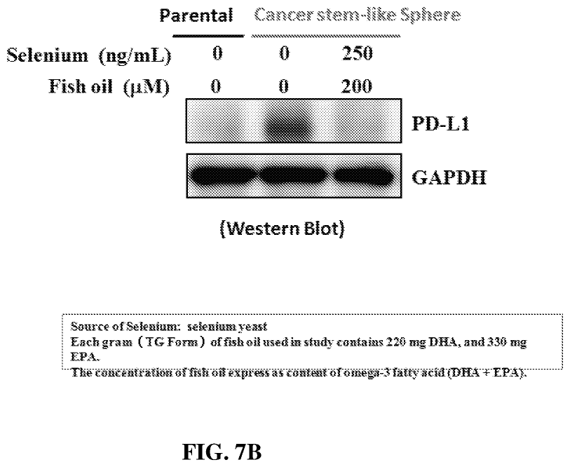

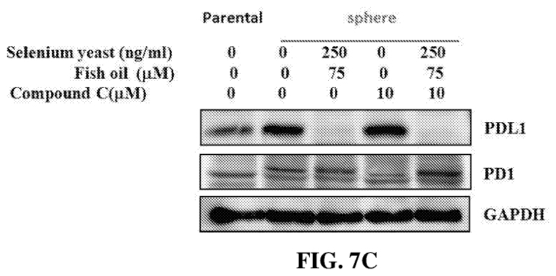

PD-1 and PD-L1 are ‘checkpoint’ proteins that regulate T-cell response and are targeted in cancer immunotherapy. PD-L1 is commonly overexpressed in cancer cells and binds to PD-1 present on activated cytotoxic T-cells, resulting their inhibition. These inhibited T-cells are ineffective in attacking the cancer cells. Immunotherapies are directed to either preventing or blocking the interaction between PD-1 and PD-L1. For example, the immunotherapeutic drug pembrolizumab is a monoclonal antibody directed to PD-1. This prevents binding of PD-L1 and blocks the inhibition of cytotoxic T-cells. Such monoclonal antibody drugs are administered by injection or infusion and are potentially immunogenic. Surprisingly, as shown in FIG. 7A the Inventors have found that a nutritional supplement that includes selenium and fish oil can dramatically reduce PD-L1 expression in HCC827 human lung cancer cells.

As shown in FIG. 6B, a combination of fish oil and selenium is effective in reducing PD-L1 expression in chemotherapy resistant cells. Surprisingly, treatment with a combination of fish oil and selenium yeast is highly effective in reducing expression of PD-L1 in such chemotherapy resistant cancer cells, whereas treatment with the chemotherapy drug Iressa has no effect.

Inventors have also found that fish oil and selenium in combination can modulate PD-L1 in cancer sphere cells, which have cancer stem cell characteristics. FIG. 7B depicts an exemplary Western blot of PD-L1 expression in stem-cell like sphere cells derived from A549 human lung cancer cells. FIG. 7C shows typical results of a similar study using different amounts of selenium and fish oil, with and without cotherapy using an antineoplastic compound. As shown PD-L1 is minimal in parental cells without stem cell characteristics, but is prominently expressed in stem-cell like sphere cells. Use of a chemotherapy drug alone fails to modulate PD-L1 expression. Application of selenium and fish oil in combination reduces PD-L1 expression levels in these stem-like sphere cells, and can reduce them to below levels observed in parental cells that do not display stem cell characteristics.

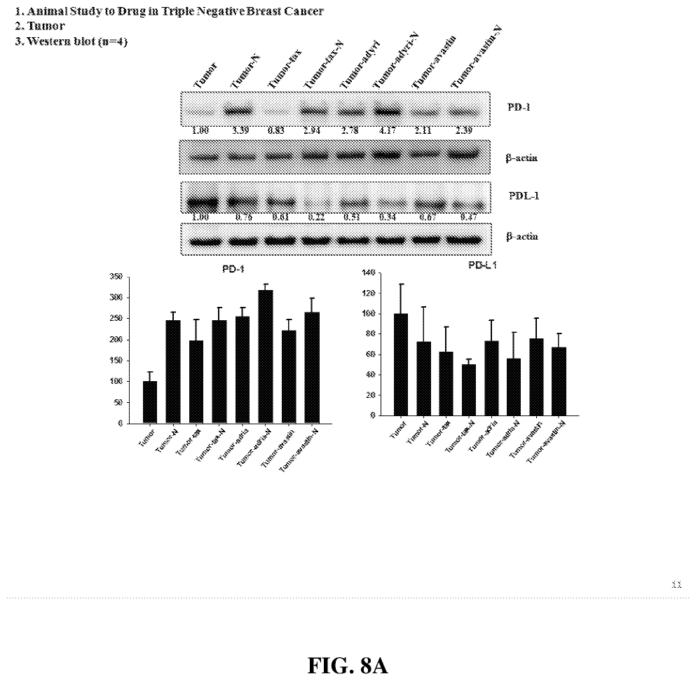

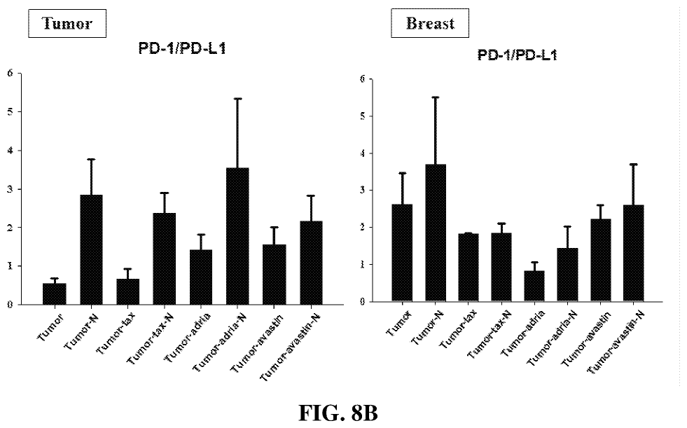

FIG. 8A show the results of studies of the effect of a nutritional supplement containing selenium and fish oil on PD-1 and PD-L1 expressed in metastatic tumor tissue (i.e. from implantation site to the breast) resulting from implantation of triple negative breast cancer cells into mice. Animals were also treated with taxol, taxol and the supplement in combination, adriamycin, adriamycin and the supplement in combination, avastin, and avastin and the supplement in combination. As shown, in vivo treatment with a nutritional supplement containing selenium and fish oil reduces expression of both PD-1 and PD-L1 in tumor tissue. Synergistic effects when used in combination with chemotherapeutic drugs are apparent. FIG. 8B shows the results of the effects of a nutritional supplement that includes selenium and fish oil on the ratio of PD-1 to PD-L1 expression in both primary tumor sites and metastatic tumor sites in an animal model of triple negative breast cancer. Synergistic effects when used in combination with chemotherapeutic drugs are apparent.

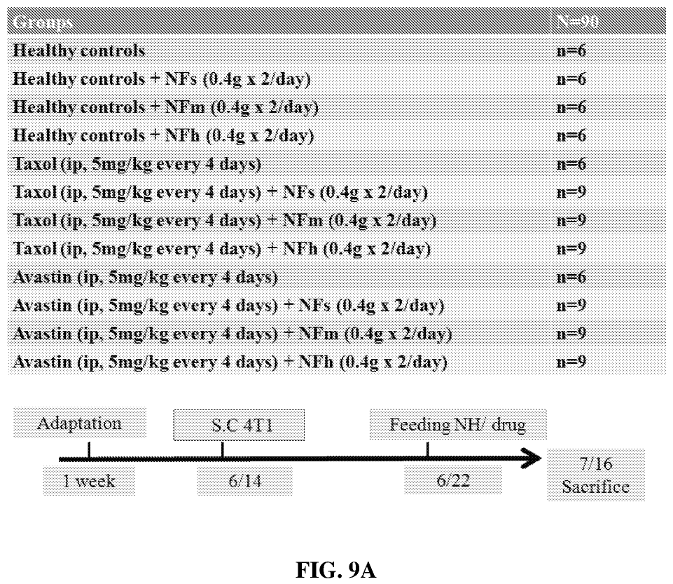

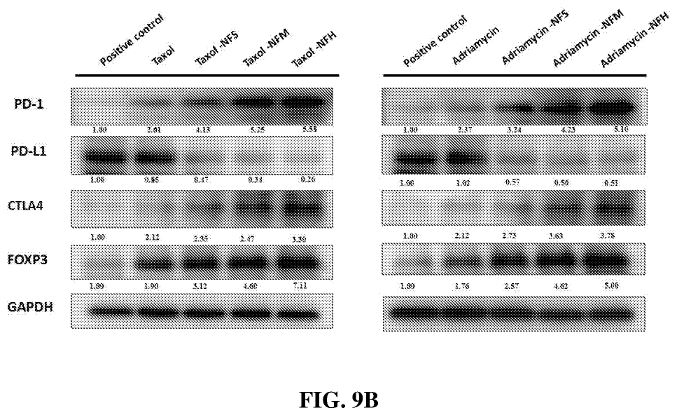

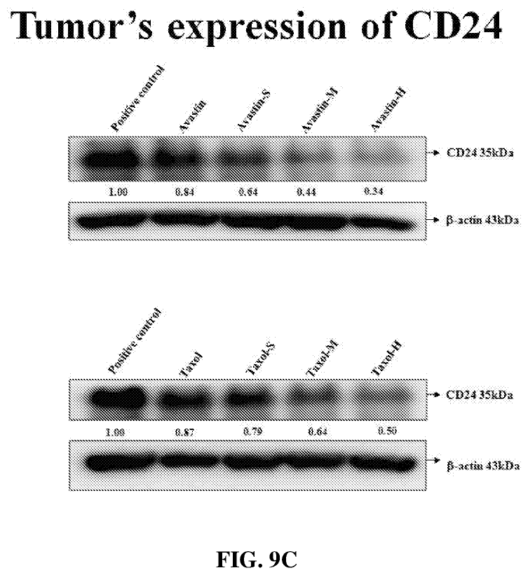

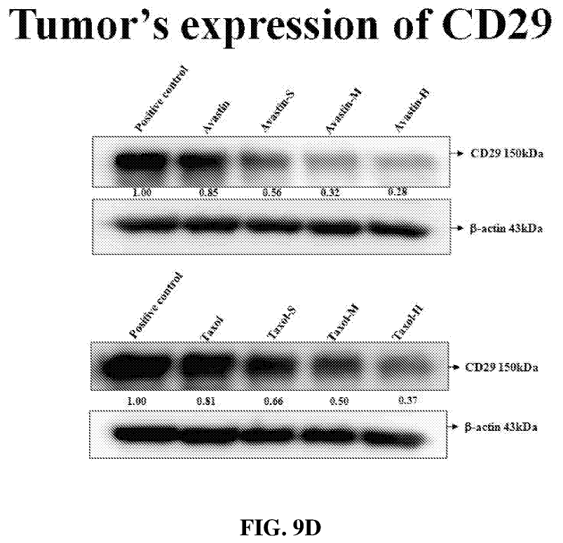

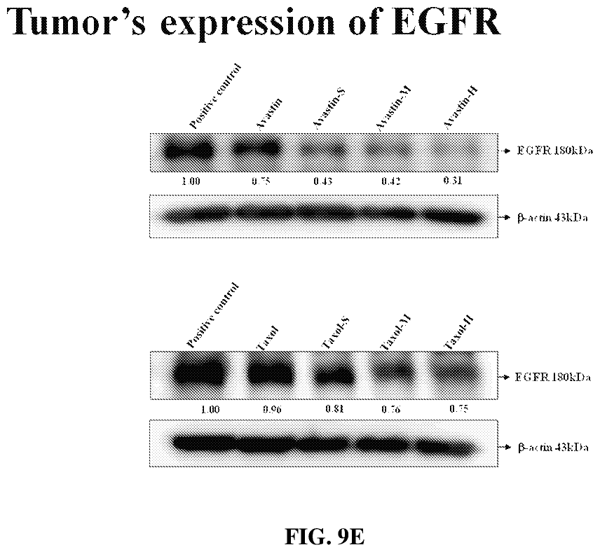

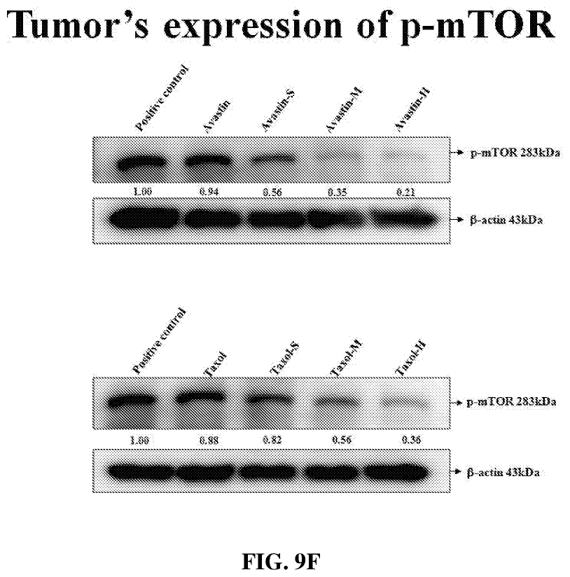

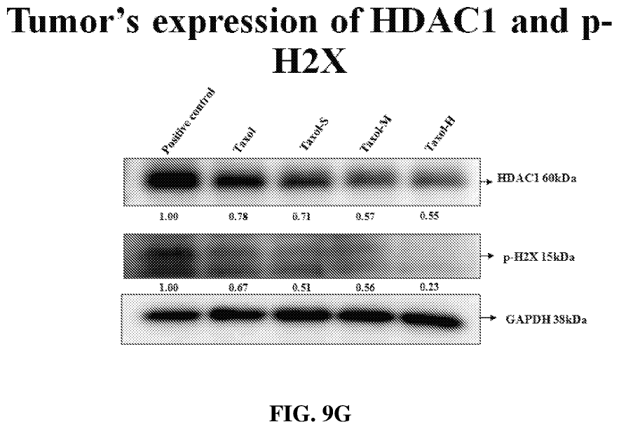

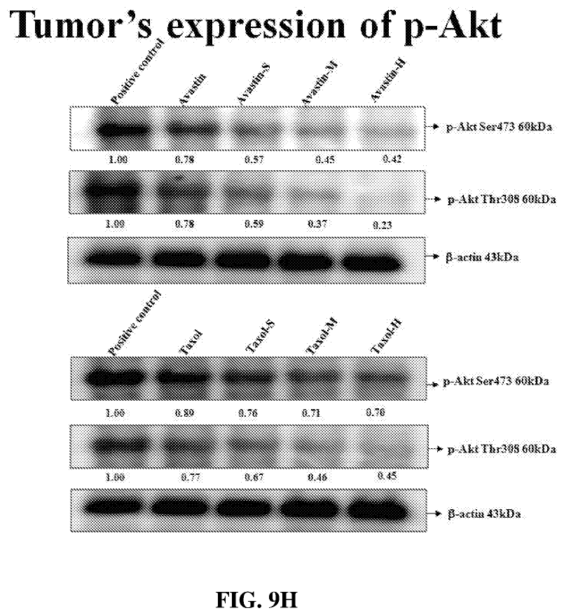

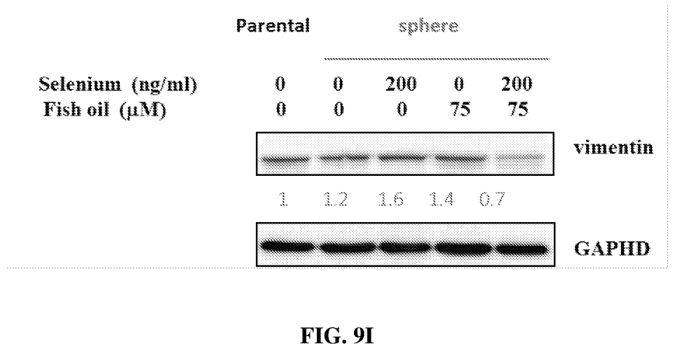

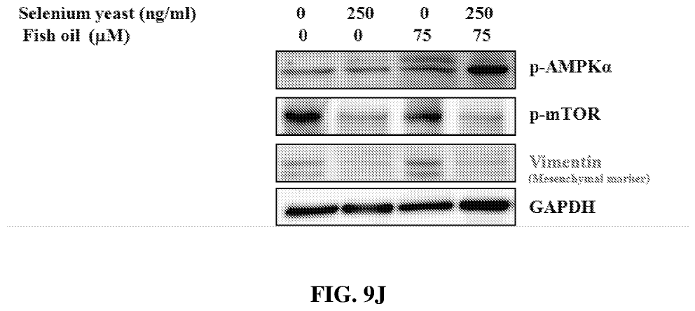

Additional studies have shown that the in vivo modulation of PD-1 and PD-L1 expression in tumor tissue and other immune checkpoint markers useful in immunotherapy by a nutritional supplement containing selenium and fish oil is dose dependent. It should be appreciated that in some embodiments a nutritional supplement of the inventive concept can provide differential effects on the expression of such immune checkpoint markers that are dependent upon the tumor location. For example modulation of immune checkpoint proteins by a nutritional supplement of the inventive concept can differ between a primary tumor site (e.g. at the site of implantation in an animal model) and a metastatic site (e.g. lung, liver, spleen, etc. in an animal model). FIG. 9A depicts a typical study design for animal model studies of this phenomena. Three different supplement formulations were used that provided low (NFS), mid (NFM-containing 1.5 times the Se content of NFS), and high (NFH, containing twice the Se content of NFS) doses of selenium and fish oil. These supplement formulations were provided in combination with a chemotherapeutic drug (such as Taxol or Adriamycin). FIG. 9B shows the results of studies of PD-1, PD-L1, CTLA4 and FOXP3 in triple negative human breast cancer primary sites in murine models. As shown, PD-L1 is reduced by use of the nutritional supplement in a dose-dependent manner, where taxol and adriamyxin have no apparent effect. Expression of PD-1, CTLA4, and FOXP3 immune checkpoint markers is increased in a dose dependent manner. FIG. 9C shows the dose dependence of the modulation of tumor CD24 expression using different levels of selenium and fish oil in combination with Avastin or Taxol in an animal tumor model. FIG. 9D shows the dose dependence of the modulation of tumor CD29 expression using different levels of selenium and fish oil in combination with Avastin or Taxol in an animal tumor model. FIG. 9E shows the dose dependence of the modulation of tumor EGFR expression using different levels of selenium and fish oil in combination with Avastin or Taxol in an animal tumor model. FIG. 9F shows the dose dependence of the modulation of tumor p-mTOR expression using different levels of selenium and fish oil in combination with Avastin or Taxol in an animal tumor model. FIG. 9G shows the dose dependence of the modulation of tumor HDAC1 and p-H2× expression using different levels of selenium and fish oil in combination with Taxol in an animal tumor model. FIG. 9H shows the dose dependence of the modulation of tumor p-Akt expression using different levels of selenium and fish oil in combination with Avastin or Taxol in an animal tumor model.

Vimentin, which can be expressed both intracellularly as well as at the cell surface, is another protein targeted by immunotherapies. As shown in FIG. 9I, combined use of fish oil and selenium reduces vimentin expression in treated cells. This effect is not seen when either fish oil or selenium is used alone. Another target of immunotherapy is p-AMPKα, which inhibits mTOR and is associated with oxidative metabolism in tumor cells and T-cells. As shown in FIG. 9J a combination of fish oil and selenium provides a synergistic effect in increasing p-AMPKα. This in turn is associated with a reduction in p-mTOR in cells so treated.

Inventors believe that use of a nutritional supplement that includes selenium and fish oil can replace and/or complement cancer chemotherapy and/or conventional immunotherapy by at least reducing tumor PD-L1 expression. It should also be appreciated that such a nutritional supplement is conveniently orally administered and is well tolerated.

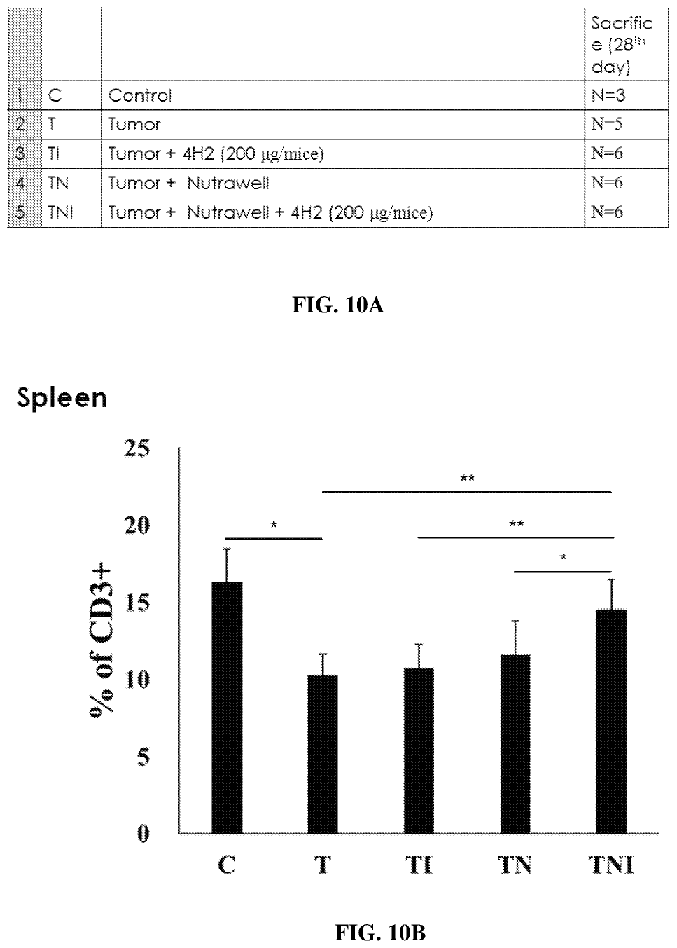

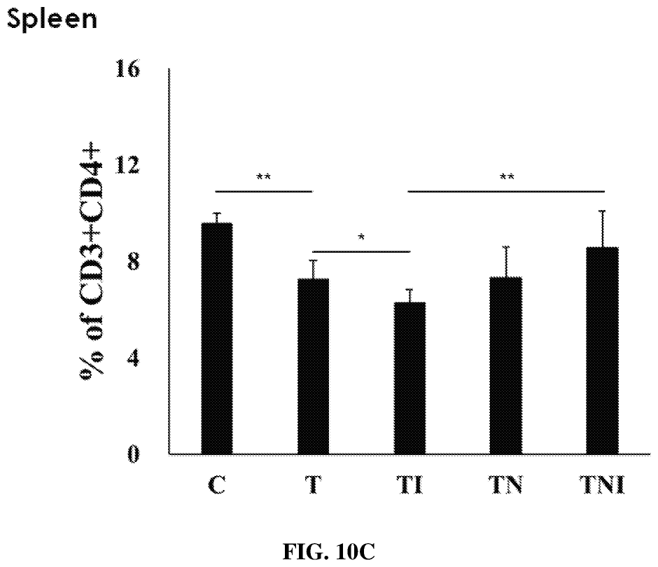

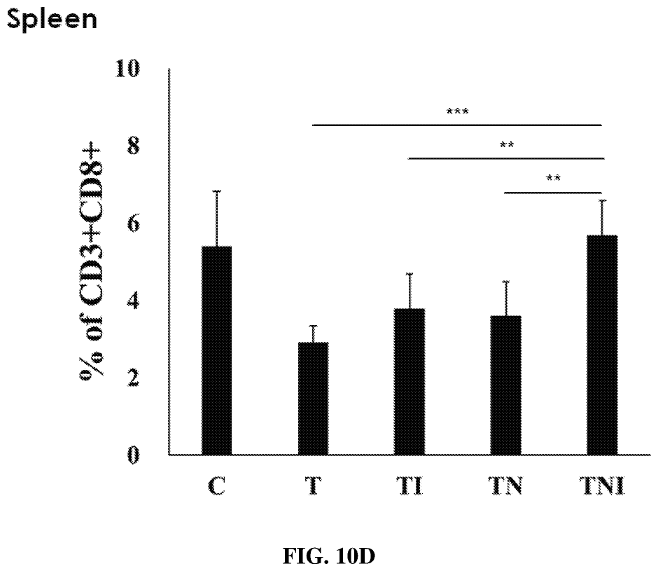

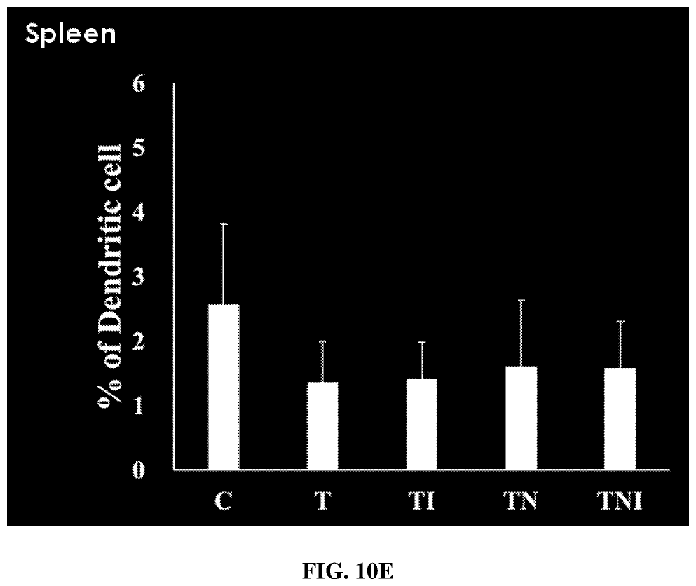

Immunotherapies involving the use of specific monoclonal antibodies (or derivatives thereof) are finding increasing use in cancer treatment. Many of these immunotherapies target the interaction between PD-1 and PDL-1. For example, 4H2 is a chimeric murine antibody specific for PD-1. FIG. 10A depicts the design of a study to determine the effect of a nutritional supplement containing fish oil and selenium when used in combination with such an antibody-based anti-tumor immunotherapy. The percentage of CD3 positive T cells found in the spleens of mice implanted with tumor cells and treated as shown in FIG. 10A are shown in FIG. 10B. As shown, untreated tumor-bearing mice show a sharply reduced number of CD3+ T cells relative to untreated controls. Treatment with the H2 antibody provided little to no improvement. Treatment with a nutritional supplement containing fish oil and selenium provided an observable improvement in the percentage of CD3+ positive T cells. Surprisingly, the combined use of a PD-1 specific antibody and a nutritional supplement containing fish oil and selenium provided a large improvement in the percentage of CD3+ T cells, indicating a synergistic effect.

The percentage of CD3 positive and CD4+ T cells found in the spleens of mice implanted with tumor cells and treated as shown in FIG. 10A are shown in FIG. 10C. As shown, untreated tumor-bearing mice show a sharply reduced percentage of CD3+/CD4+ T cells relative to untreated controls. Treatment with the H2 antibody appears to reduce the percentage of CD3+/CD4+ T cells even further. Treatment with a nutritional supplement containing fish oil and selenium has little impact on the percentage of CD3+/CD4+ positive T cells. Surprisingly, the combined use of a PD-1 specific antibody and a nutritional supplement containing fish oil and selenium provided an improvement in the percentage of CD3+/CD4+ T cells, indicating a synergistic effect.

The percentage of CD3 positive and CD8 positive T cells found in the spleens of mice implanted with tumor cells and treated as shown in FIG. 10A are shown in FIG. 10D. As shown, untreated tumor-bearing mice show a sharply reduced percentage of CD3+/CD8+ T cells relative to untreated controls. Treatment with the H2 antibody slightly increases the percentage of CD3+/CD8+ T cells, as does treatment with a nutritional supplement containing fish oil and selenium. Surprisingly, the combined use of a PD-1 specific antibody and a nutritional supplement containing fish oil and selenium provided a dramatic improvement in the percentage of CD3+/CD8+ T cells, indicating a synergistic effect.

The percentage of dendritic cells found in the spleens of mice implanted with tumor cells and treated as shown in FIG. 10A are shown in FIG. 10E. As shown, untreated tumor-bearing mice show a sharply reduced percentage of dendritic cells relative to untreated controls. Treatment with the H2 antibody has little effect on the percentage of dendritic cells. Treatment with a nutritional supplement containing fish oil and selenium provides an increase in the percentage of dendritic cells, which is also found with the combined use of a PD-1 specific antibody and a nutritional supplement containing fish oil and selenium.

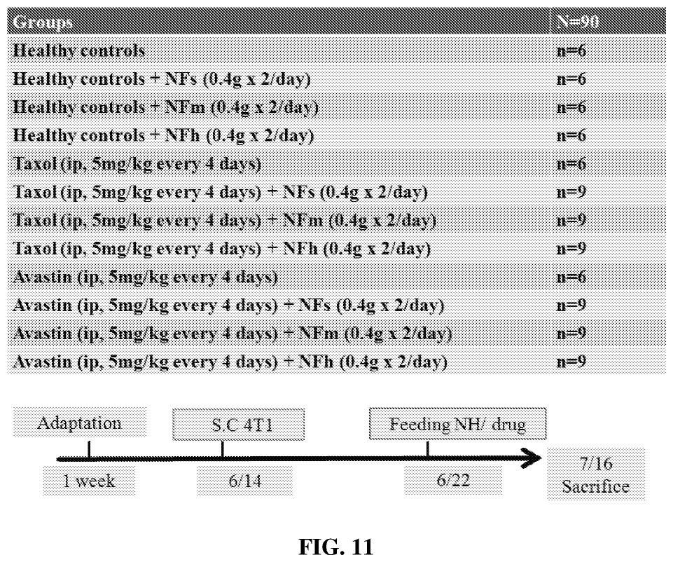

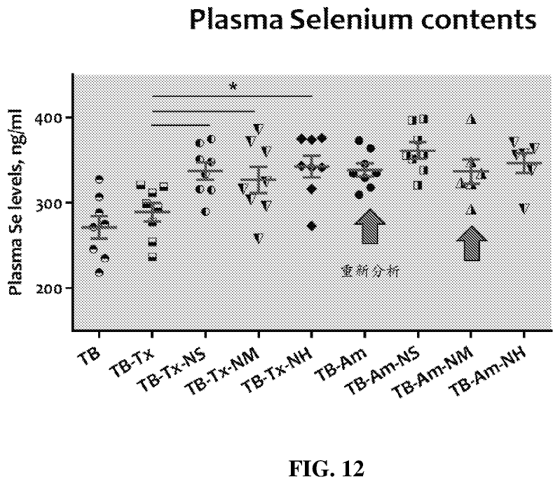

As noted above, nutritional supplements can be provided that include different amounts of selenium (Se), and can also be provided with or used in combination with a chemotherapeutic agent. A typical dosing schedule used in animal studies is shown in FIG. 11. FIG. 12 shows the effects of such a dosing schedule on serum/plasma concentrations of selenium in tumor-bearing mice. As shown in FIG. 12, treatment of tumor-bearing mice with a nutritional supplement containing selenium and fish oil along with treatment with a chemotherapeutic agent resulted in elevated plasma selenium concentrations. Notably, plasma selenium concentrations were relatively consistent despite treatment with nutritional supplements having a range of selenium content.

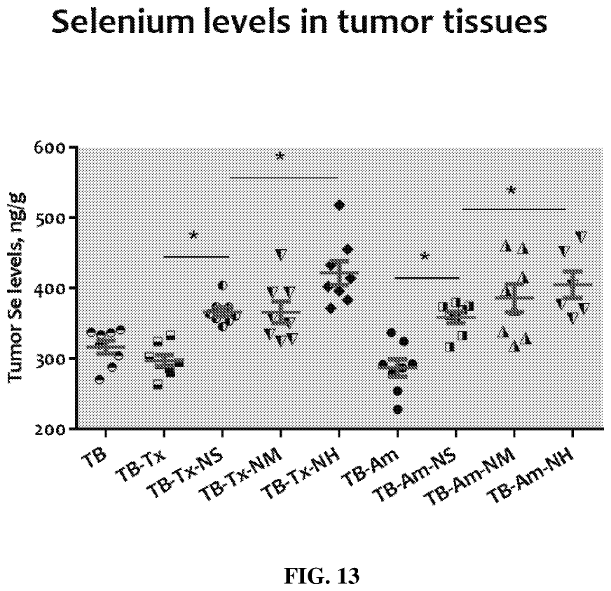

FIG. 13 shows selenium content of tumor tissue obtained from tumor-bearing mice treated as in FIG. 12. Surprisingly, selenium is sequestered in a selenium dose-related manner in tumor tissue of tumor-bearing mice treated with a combination of a nutritional supplement containing fish oil and selenium and also treated with a chemotherapeutic agent. This indicates selective uptake and/or retention of selenium, potentially providing localized effects in tumor tissue while being provided systemically through simple oral administration.

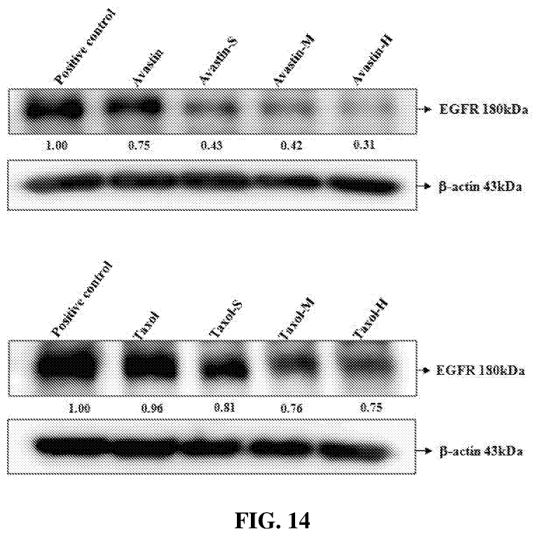

Epidermal growth factor receptor (EGFR) is a driver of tumorigenesis, and is frequently inappropriately activated (for example, by amplification) in lung cancer, breast cancer, and glioblastoma cells. EGFR is also associated with the development of drug resistance, as amplification has been shown to be driven by selective pressure applied by chemotherapeutic drugs. As shown in FIG. 14, EGFR expression is reduced in tumor tissues in a dose-specific manner when tumor-bearing animals are treated with a nutritional supplement containing selenium in different amounts and fish oil and co-treated with a chemotherapeutic drug (e.g. taxol or avastin) Surprisingly, EGFR expression is reduced in a selenium-dose dependent manner, even when expression is not impacted by chemotherapy alone (e.g. taxol).

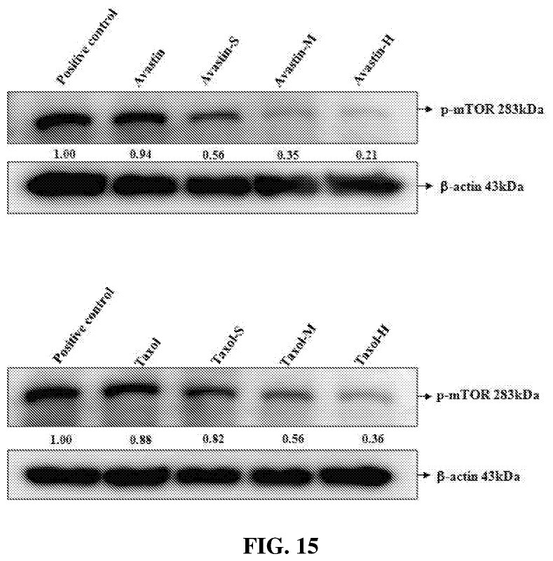

As noted above, phosphorylated mTOR (p-mTOR) is also associated with tumor growth and drug resistance. Treatment of tumor-bearing mice with a chemotherapeutic agent e.g. taxol or avastin) in combination with a nutritional supplement containing fish oil and different amounts of selenium was found to sharply decrease expression of p-mTOR in a selenium-dose dependent manner, as shown in FIG. 15.

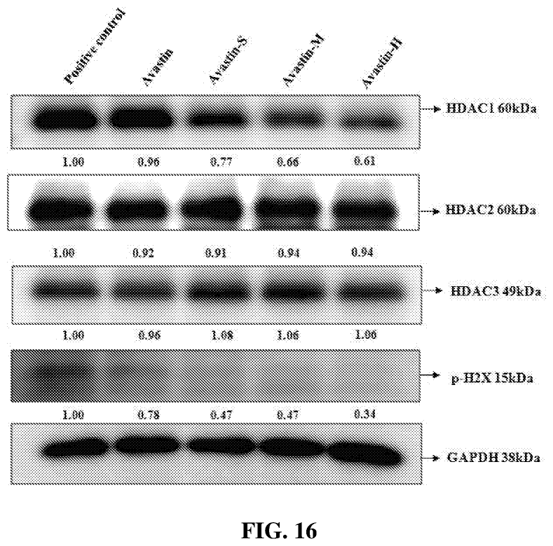

Histone acetylation mediated by histone acetyltransferase represents an epigenetic modification that impacts gene expression. Aberrant expression of histone deacetylase is linked with tumor development, with altered gene expression leading to disruption of cellular functions such as cell proliferation, cell-cycle regulation, and apoptosis. Accordingly, inhibition of histone deacetylase is being investigated as a target for cancer therapy. As shown in FIG. 16, the use of a nutritional supplement containing fish oil and different amount of selenium in combination with chemotherapeutic agents has been found to reduce histone deacetylase expression in tumors of tumor-bearing mice in a selenium-dose dependent manner. FIG. 16 also shows that p-H2X, a marker associated with the presence of double stranded DNA breaks, is strongly reduced in tumor tissue of tumor-bearing mice treated with a combination of a chemotherapeutic agent and a nutritional supplement that includes fish oil and different amounts of selenium, in a selenium-dose dependent manner.

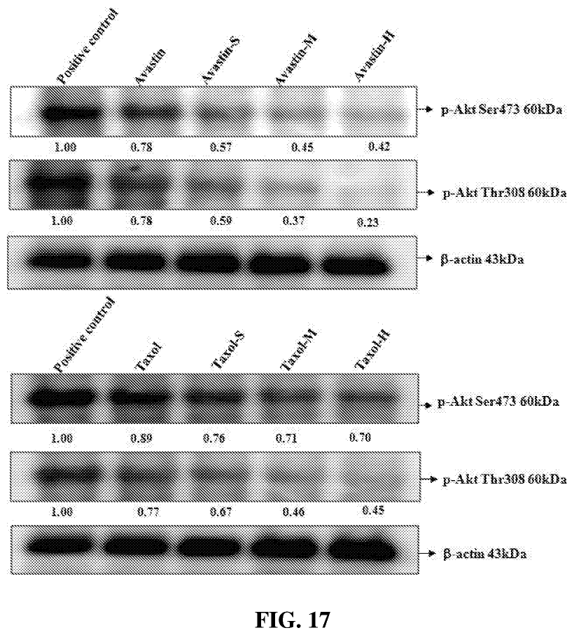

Phosphorylated Akt (p-Akt) is considered a marker for poor prognosis in some cancers, including breast and gastric cancer. As shown in FIG. 17, when tumor-bearing mice are treated with a nutritional supplement containing fish oil and selenium id different amounts in combination with a chemotherapeutic agent both p-Akt Thr308 and p-Akt Ser473 is reduced in tumor tissue. The observed reduction is selenium-dose dependent.

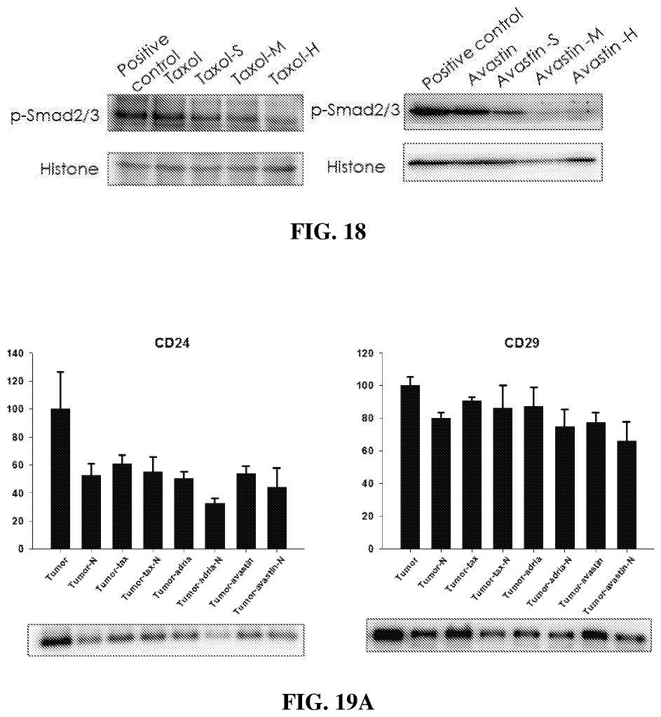

The Smad family of proteins is part of the TGF-β signaling pathway, which is involved in both the development and metastasis of tumors. Elevated levels of p-Smad 2 in the nuclei of cancer cells is associated with poor prognosis. As shown in FIG. 18, treatment of tumor-bearing mice with a nutritional supplement containing fish oil and different amounts of selenium, in combination with a chemotherapeutic agent, dramatically reduced p-Smad 2/3 content in the nuclei of tumor cells. The degree of reduction is selenium-dose dependent.

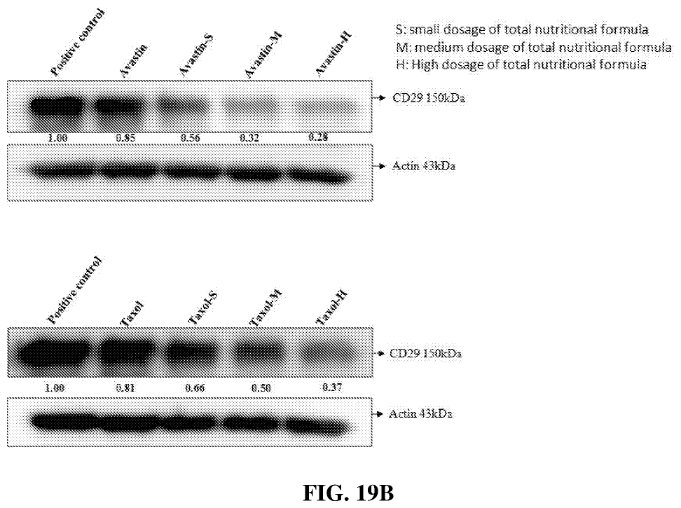

Cancer cells can have stem cell-like characteristics, the presence of which can be indicative of the ability to metastasize and/or develop resistance to chemotherapeutic drugs. CD24 and CD29 are markers that are commonly used to determine degree of stem cell character. FIG. 19A shows the results of in vivo studies of the effect of a nutritional supplement that includes selenium and fish oil of CD24 and CD29 expression in the primary tumor site produced by injection of mice with triple negative breast cancer cells. Mice were also treated with taxol, taxol and the supplement in combination, adriamycin, adriamycin and the supplement in combination, avastin, and avastin and the nutritional supplement in combination. FIG. 19B shows that modulation of tumor C24 and CD29 expression through use of a nutritional supplement that includes selenium and fish oil is dose dependent. As in FIG. 9, nutritional supplements containing low, mid, and high amounts of selenium were provided in combination with either avastin or taxol. As shown, a nutritional supplement containing selenium and fish oil can significantly reduce expression of CD24 and CD29 in primary tumor sites (in a dose dependent manner), even when conventional chemotherapeutic agents are largely ineffective. This indicates a reduced degree of stem cell-like character in the tumor cells, and subsequently a reduced tendency to metastasize and/or develop drug resistance.

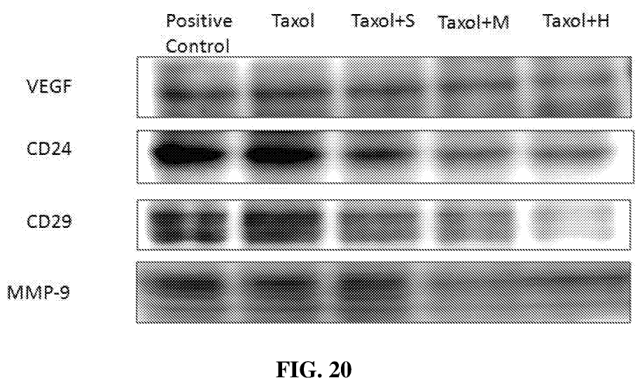

FIG. 20 shows the results of the use of different amounts of a nutritional supplement containing selenium and fish oil (provided in combination with Taxol) on markers related to metastatic capability, specifically CD24, CD29, MMP-9, and VEGF in samples obtained from a metastatic brain tumor site. While taxol alone had no apparent effect, use of a nutritional supplement containing selenium and fish oil reduced expression of VEGF, CD24, CD29, and MMP-9 markers related to metastasis in a dose dependent manner.

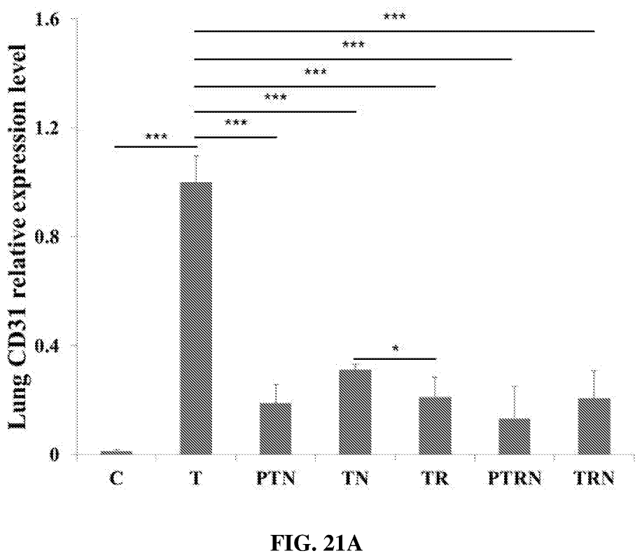

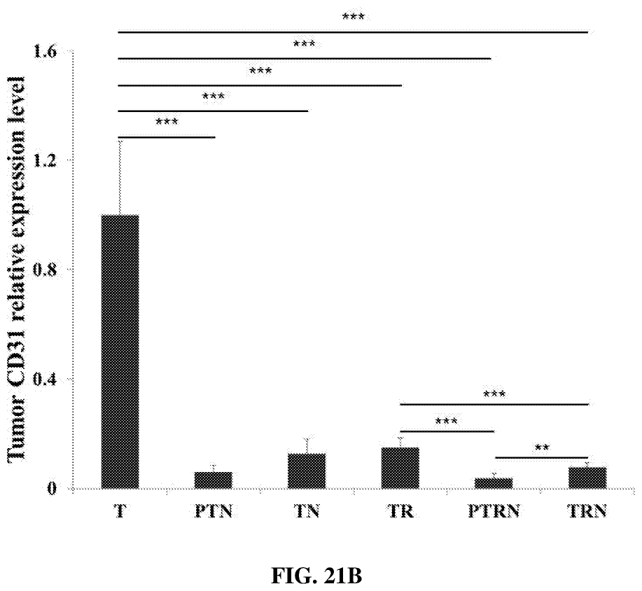

CD31 is another marker that is indicative of stem cell characteristics in tumor cells, which are related to their tendency to metastasize. FIGS. 21A and 21B show the results of in vitro studies of CD31 expression levels in metastatic and primary tumor sites for lung cancer cells implanted into mice. Animals were either pre-treated with the nutritional supplement or began treatment at the time of implantation. As shown in FIGS. 21A and 21B, a nutritional supplement containing selenium and fish oil is effective in reducing expression of CD31 in both metastatic tissue and primary tumor tissue, with pre-treatment providing effects similar to that of conventional radiotherapy (but without the attendant side effects).

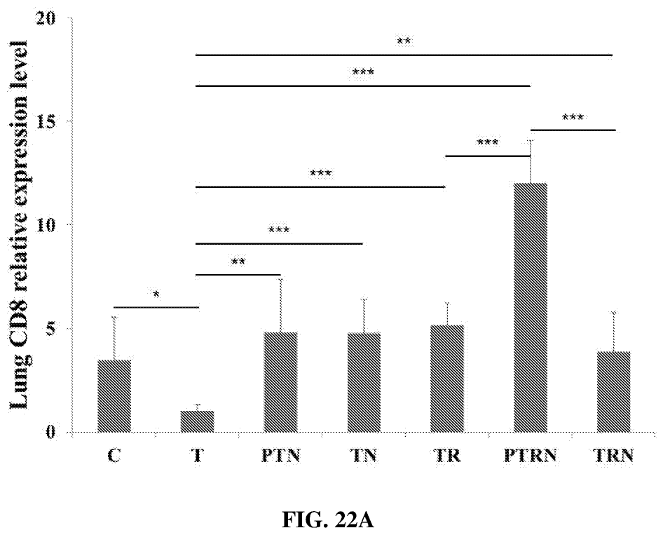

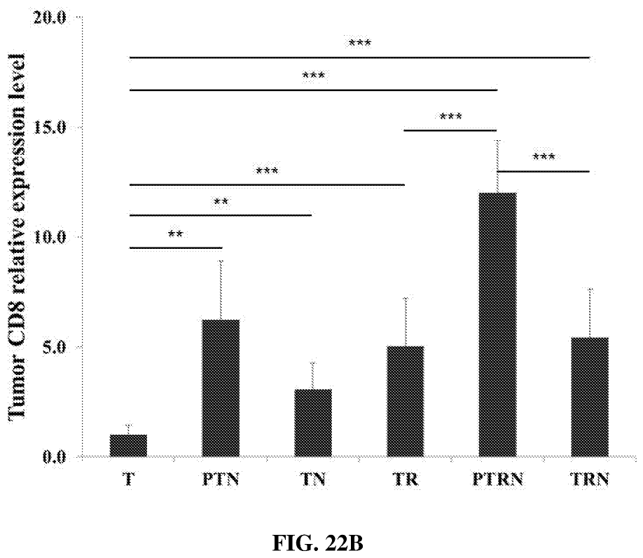

CD8 positive T cells are another potential target for cancer immunotherapy. The presence of such tumor infiltrating T cells in tumors correlates with overall survival in cancer patients. FIGS. 22A and 22B show the results of in vitro studies of CD8 expression levels in metastatic and primary tumor sites for lung cancer cells implanted into mice. Animals were either pre-treated with the nutritional supplement or began treatment at the time of implantation. As shown in FIGS. 22A and 22B, a nutritional supplement containing selenium and fish oil is effective in increasing infiltration of CD8 positive T cells in both metastatic tissue and primary tumor tissue, with pre-treatment providing effects similar to that of conventional radiotherapy (but without the attendant side effects). Surprisingly, a significant synergistic effect is found when pre-treatment with the nutritional supplement is combined with radiotherapy.

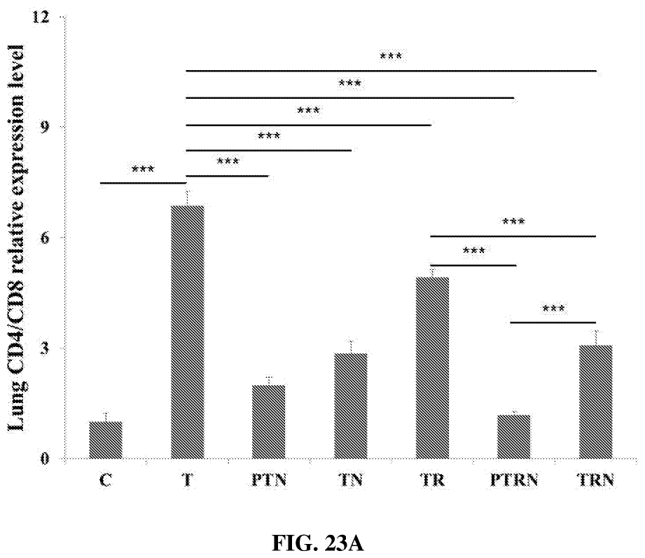

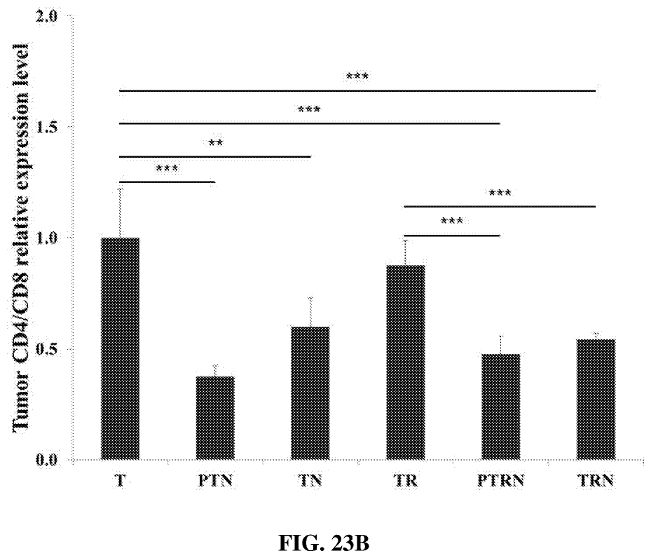

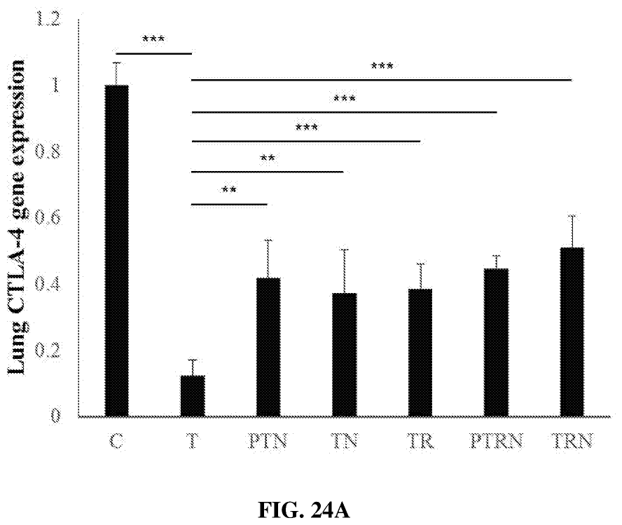

CD4 positive T cells, which are active towards pre-oncogenic senescent cells, are another target for cancer immunotherapy. Such CD4 positive T cells act in concert with CD8 positive T cells, with CD8 positive T cells becoming active as senescent cells accumulate further mutations and move from senescence to oncogenesis. As such a normal balance between CD4 positive and CD8 positive T cells is desirable. The ratio between CD4 and CD8 expression provides a measure of the relative populations of these T cells in affected tissues. FIGS. 23A and 23B show the results of in vitro studies of CD4 expression relative to CD8 expression levels in metastatic and primary tumor sites for human lung cancer cells implanted into mice. Animals were either pre-treated with the nutritional supplement or began treatment at the time of implantation. As shown in FIGS. 23A and 23B, a nutritional supplement containing selenium and fish oil is effective in reducing the CD4/CD8 ratio in both metastatic tissue and primary tumor tissue, with pre-treatment providing effects similar to that of conventional radiotherapy (but without the attendant side effects). Surprisingly, treatment with the nutritional supplement (particularly pre-treatment) was more effective than conventional radiotherapy.

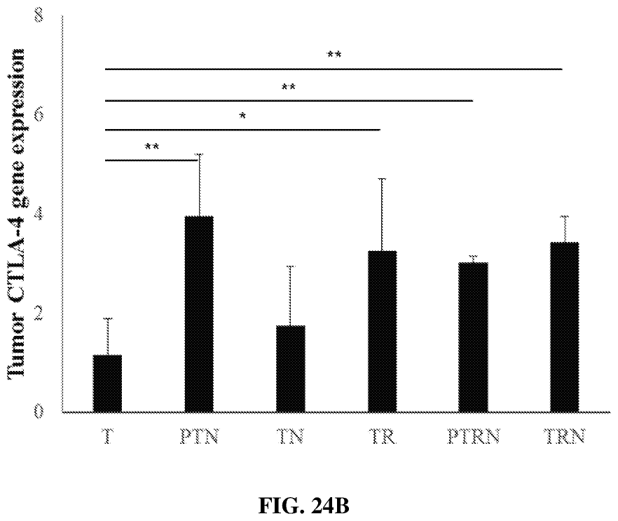

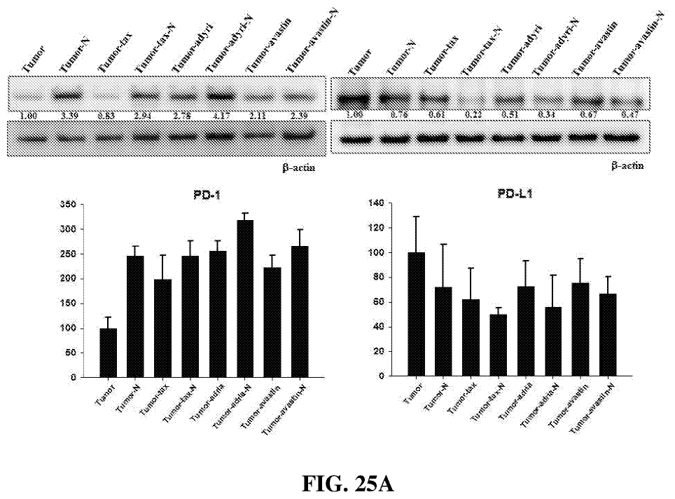

CTLA4 is another immune checkpoint protein that downregulates immune responses, and is a potential target in cancer immunotherapies. For example, ipilimumab is a therapeutic antibody directed to CTLA4 that is used in cancer immunotherapy. Unfortunately use of ipilimumab can result in severe autoimmune effects. FIGS. 24A and 24B show the results of in vitro studies of CTLA4 expression levels in metastatic and primary tumor sites for human lung cancer cells implanted into mice. Animals were either pre-treated with the nutritional supplement or began treatment at the time of implantation. As shown in FIGS. 24A and 24B, a nutritional supplement containing selenium and fish oil is effective in increasing CTLA4 expression in both metastatic tissue and primary tumor tissue, with pre-treatment providing effects similar to that of conventional radiotherapy (but without the attendant side effects).

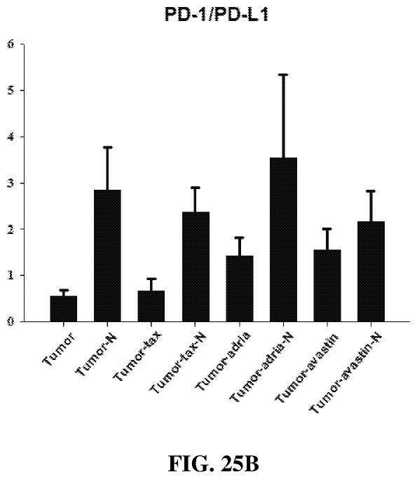

In some embodiments of the inventive concept a nutritional supplement containing selenium and fish oil can be used to modify or regulate PD-1 and/o r PDL-1 expression FIG. 25A shows the results of treatment of PD-1 and PDL-1 expression in animal models of breast cancer models when treated with a nutritional supplement containing selenium and fish oil and commonly used chemotherapeutic agents. FIG. 25B shows the ratio between PD-1 and PD-L1 expression in these tumors. As shown, treatment with a nutritional supplement containing selenium and fish oil reduces PD-L1 expression in tumors. This decrease at least complements reductions in PD-L1 observed on treatment with chemotherapeutic agents. Conversely, expression of PD-1 is increased by treatment with a nutritional supplement containing selenium and fish oil. It is apparent that the ratio of PD-1 to PD-L1 expression is low in untreated tumor, but dramatically increased on treatment with a supplement containing selenium and fish oil. Treatment with chemotherapeutic agents had a relatively small effect on the PD-1:PD-L1 ratio, however this was enhanced by cotherapy with the nutritional supplement.

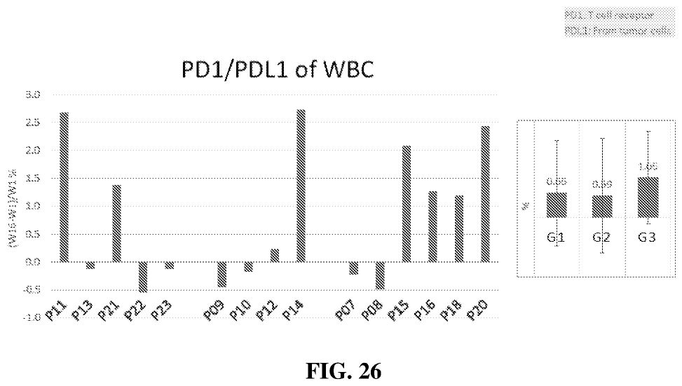

The effects of a nutritional supplement containing selenium and fish oil on the rate of change in the ratio of PD1 content of white blood cells to PDL-1 content of tumor cells has also been studied in human clinical trials. FIG. 26 shows the result of such a trial, in which patients received a low dose supplement (G1), a mid level dose supplement (G2, having 1.5 times the Se content of G1), or a high dose supplement (G3, containing twice the Se content of G1). PD1 content of white blood cells and PDL-1 content of tumor cells was determined at week 1 and week 16 of treatment. As shown, providing a nutritional supplement containing selenium and fish oil to patients with cancer can increase the white blood cell PD1/tumor PDL-1 ratio, and does so in a dose-dependent manner.

Human clinical trials were also performed to characterize the effect of a nutritional supplement containing selenium and fish oil on the number of circulating tumor cells (CTC), which is related to metastasis from the primary tumor site. Patients were separated into three groups that received different amounts of the nutritional supplement, and the number of circulating tumor cells, serum selenium content, serum EPA content, and serum DHA content were characterized. Results are shown in Table 2.

TABLE 2

| | | | | | [symbol] | | [symbol] | [symbol] | [symbol] |
|---|---|---|---|---|---|---|---|---|---|
| Patent | | [symbol] | | [symbol] | [symbol] | | [symbol] | [symbol] | [symbol] |
| Number | Group | [symbol] | Cancer type | [symbol] | [symbol] | Change of CTC | [symbol] | [symbol] | [symbol] |
| G1-1 | G1 | Oral | Lung adenocarcinoma | Verified after | 53% | [symbol] | 98.8 | 16.1 | 226.9 |
| | | | meta liver, spleen | completed | | | | | |
| G1-2 | G1 | Oral | Hypophagryngeal ca | V | 53% | [symbol] | 91.5 | 7.2 | 240.2 |
| G1-3 | G1 | Oral | Lung ca | V | 53% | [symbol] | 125.4 | 36.3 | 341.1 |
| G1-4 | G1 | | Lung ca | V | | | | | |
| G1-5 | G1 | | Head and neck ca | V | | | | | |
| G1-6 | G1 | | Lung ca | V | | | | | |
| G2-1 | G2 | [symbol] | Ca of chest | Verified after | 100% | [symbol] | 154.5 | 68.9836 | 330.5 |
| | | | | completed | | | | | |
| G2-2 | G2 | Oral | NPC | Verified after | 53% | [symbol] | 326.9 | 100.336 | 407.8 |
| | | | Oral cavity ca | completed | | | | | |
| G2-4 | G2 | Oral | meta liver | Verified after | 13% | [symbol] | 73.2 | 20.8531 | 309.8 |
| | | | | completed | | | | | |
| G2-5 | G2 | Oral | NPC | V | 53% | [symbol] | 94.8 | 24.1216 | 227.5 |
| | | | meta pancreas | | | | | | |
| G2-6 | G2 | NG | Oral cavity ca | Verified after | 100% | [symbol] | 158.7 | 119.947 | 392.1 |
| | | | | completed | | | | | |
| G2-7 | G2 | Oral | Lung ca | V | 40% | [symbol] | 139.5 | | |
| G2-8 | G2 | | Esophageal ca | V | | | | | |
| G3-2 | G3 | NG | NPC | Verified after | 100% | [symbol] | 692.8 | 226.172 | 401.0 |
| | | | | completed | | | | | |
| G3-3 | G3 | Oral | Carcinoma of gingiva | Verified after | 44% | [symbol] | 318.5 | 63 | 354.3 |
| | | | | completed | | | | | |
| G3-4 | G3 | Oral | Lung adenocarcinoma | Verified after | 66% | [symbol] | 473.4 | 142.803 | 422.9 |
| | | | meta bone, adrenal gland | completed | | | | | |
| G3-5 | G3 | Oral | Lung ca | V | 22% | [symbol] | 105.4 | | |
| G3-6 | G3 | Oral | Lung ca | [symbol] | 11% | [symbol] | | | |
| | | | | [symbol] | | | | | |
| G3-7 | G3 | Oral | Lung ca | V | 33% | [symbol] | 173.6 | 73.8 | 495.9 |
| G3-8 | G3 | Oral | Head and neck ca | [symbol] | 82% | [symbol] | | | |
| | | | | [symbol] | | | | | |
| G3-9 | G3 | | Lung ca | | | [symbol] | | | |
| G3-10 | G3 | | Head and neck ca | | | | | | |

Group 1 (G1) patients received low doses of' a nutritional supplement containing selenium and fish oil, group 2 (G2) patients received medium doses of' the supplement, and group 3 ((13) patients received high doses of' the supplement. [symbol]
indicates data missing or illegible when filed

As shown in Table 2, patients receiving the highest dose of a nutritional supplement containing selenium and fish oil showed the highest rate of reduction in circulating tumor cells, and also showed the highest serum concentrations of selenium, EPA, and DHA. Accordingly, providing cancer patients with a supplement that includes selenium and fish oil can result in a reduction in circulating tumor cells and, consequently, reduced tumor metastasis.

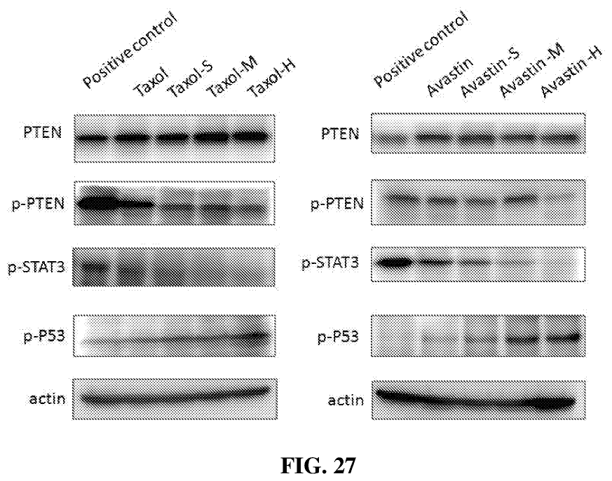

Inventors have also found that a supplement containing selenium and fish oil can enhance NK cell activity in animal models of breast cancer. FIG. 27 shows the results of studies of such a supplement provided at low, mid, and high doses in combination with chemotherapeutic drugs (i.e. taxol and avastin). As shown expression of the tumor suppressor PTEN is increase in a dose-dependent manner over the increase induced by taxol. Expression of p-PTEN is decreased in a dose-dependent manner over the decrease induced by either taxol or avastin. The expression of p-STAT3 is similarly reduced in a dose-dependent manner over the decrease induced by either taxol or avastin, and is decreased to nearly undetectable levels at high doses of a nutritional supplement containing selenium and fish oil. Expression of phospho-P53, which is low to nearly undetectable in untreated animal subjects, is increased in a dose-dependent manner over the effects produced by either taxol or avastin.

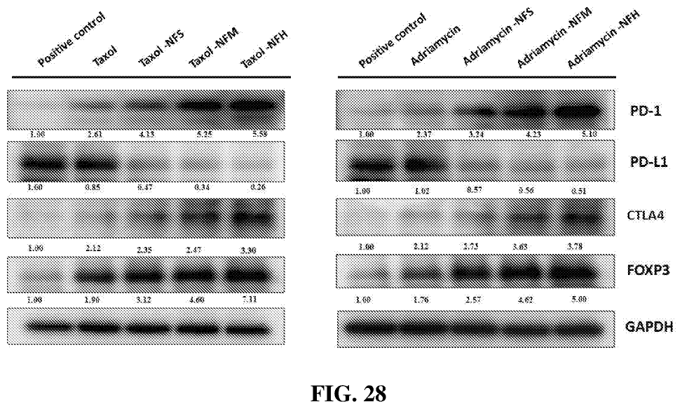

A similar study was performed in which T-cell and immune checkpoint markers were characterized in samples taken from triple negative breast cancer tumors in an animal model of human disease. Results are shown in FIG. 28. As shown expression of the immune checkpoint protein PD-1 is increased slightly by treatment with taxol or adriamycin, and this is enhanced in a dose-dependent manner by treatment with a nutritional supplement containing selenium and fish oil. The Inventor believes that this is at least in part due to more extensive T-cell infiltration of tumor tissue, due to enhanced activity. Conversely, expression of the immune checkpoint protein PD-L1 is essentially unaffected by treatment with chemotherapeutic agents, and is decreased dramatically and in a dose-dependent manner by cotherapy with a nutritional supplement containing selenium and fish oil. Expression of both CTLA4 and FOXP3 are increased by treatment with chemotherapeutic agents, and the effect is enhanced in a dose-dependent manner by cotherapy with the nutritional supplement.

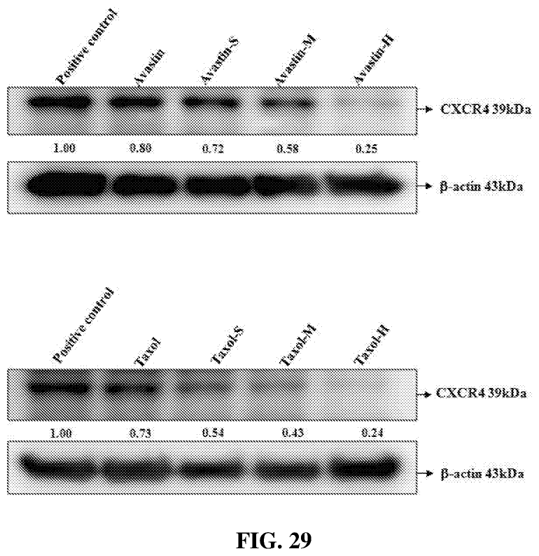

CXCR4 expression in tumors is associated with metastasis to CXCL12 expressing tissues, and CXCR4 inhibiting compounds have been shown to have anti-tumor activity. As shown in FIG. 29 (which utilized an animal model of breast cancer similar to that described in FIG. 28) treatment with avastin alone has little effect on tumor expression of CXCR4, however cotherapy with a nutritional supplement that includes selenium and fish oil results in dramatic, dose dependent decreases in CXCR4 expression in tumor tissue.

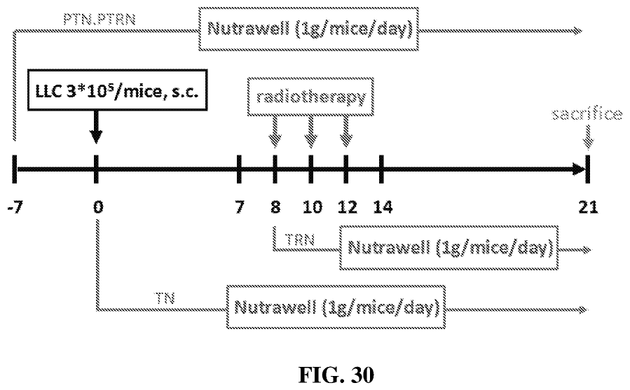

Similar studies were performed in animal models of lung cancer, where the nutritional supplement was used in combination with radiotherapy. In some experiments nutritional supplementation with a supplement containing selenium and fish oil was provided for 7 days prior to the initiation of repeated rounds of radiotherapy; in other embodiments nutritional supplementation and radiotherapy began simultaneously. A typical study is shown in FIG. 30.

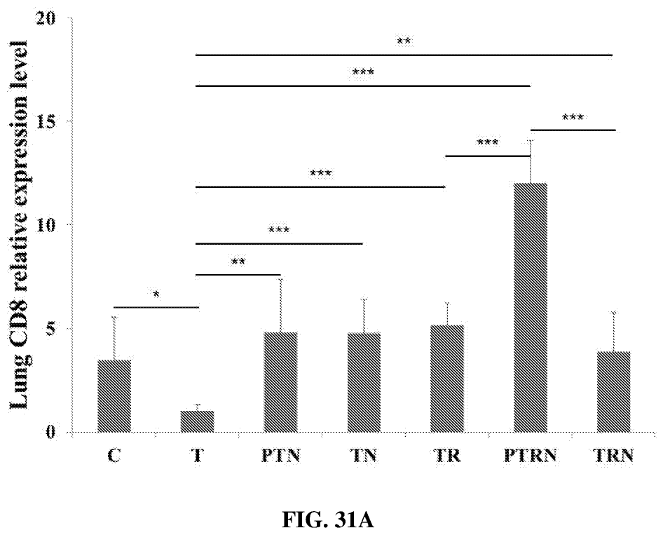

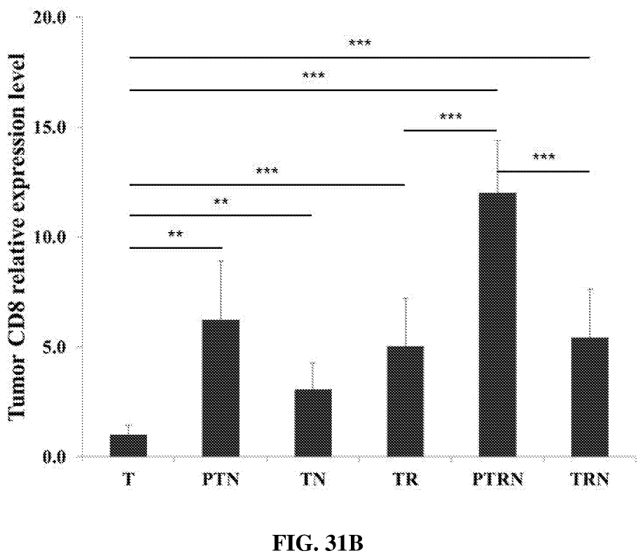

CD8 is associated with cytotoxic T-cells, with an elevated CD4/CD8 ratio being associated with increased survival. As shown in FIG. 31A expression of CD8 is decreased in samples of lung tissue if tumor-bearing animals (T) relative to control, untreated animals (C). Both pretreatment with a nutritional supplement containing selenium and fish oil (PT) and treatment on day 8 after implantation (TN) increase CD8 expression, as does radiotherapy (TR) and cotherapy with radiation and the nutritional supplement (TRN). Surprisingly, pretreatment with the nutritional supplement followed by radiotherapy in day 8 (PTRN) provided a synergistic effect in increasing CD8 expression. It should be appreciated that the results from FIG. 31A are representative of metastasis from the primary implantation site of the tumor cells to the lungs of the animal. As shown in FIG. 31B similar results are found in tumor tissue at the primary implantation site.

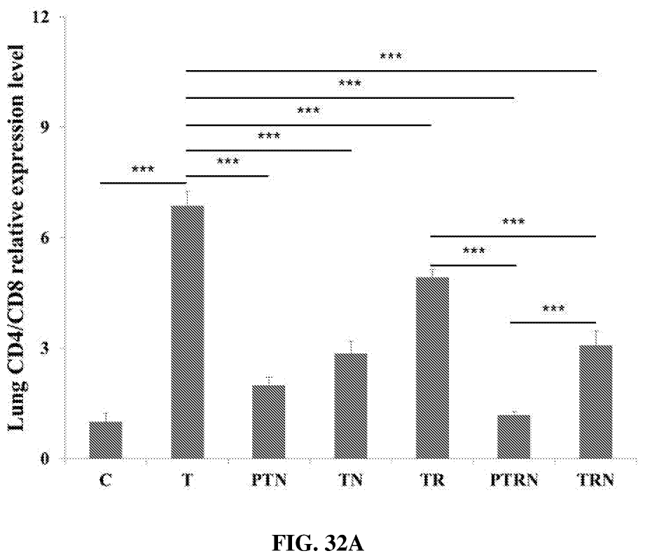

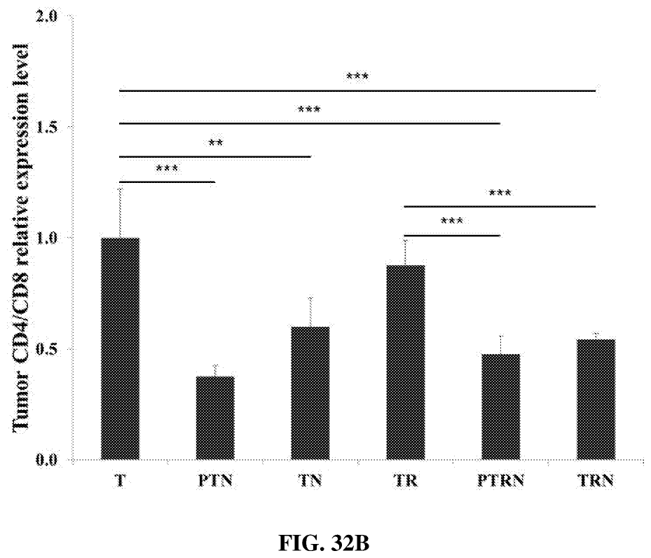

As noted above, the CD4/CD8 ratio is considered indicative of T cell activity directed to tumors. As shown in FIG. 32A, in lung tissue of an animal model of lung cancer (representing metastatic sites) the CD4/CD8 ratio of control untreated animals (C) is low relative to the ratio found in untreated animals injected with lung cancer cells (T). Using a protocol as shown above, lung tissue from animals treated only with radiation (TR) show a slight reduction in CD4/CD8 ratio, whereas animals treated with a nutritional supplement containing selenium and fish oil show marked reductions in the CD4/CD8 ratio. This was found for animals treated only with the nutritional supplement (PTN, TN) or when the nutritional supplement was provided along with radiotherapy (PTRN, TRN). It is notable that treatment with a nutritional supplement containing selenium and fish oil prior to initiation of radiotherapy (PTRN) reduced the CD4/CD8 ratio of lung tissue to essentially that of control animals. FIG. 32B shows the results of similar studies in which tissue samples were obtained from the tumors generated at the primary implantation site.

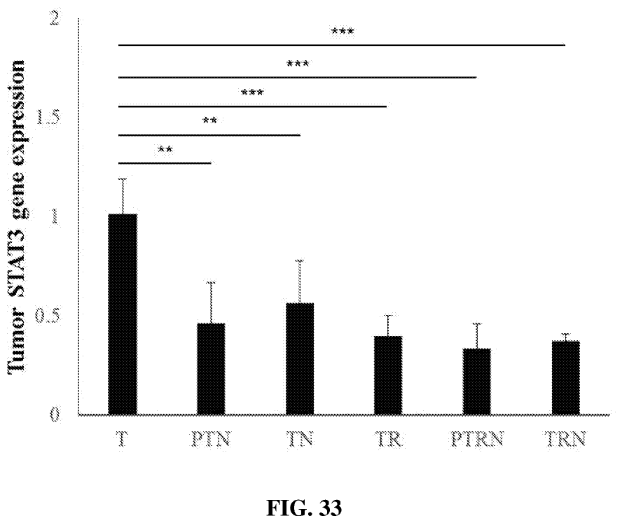

STAT3 is observed to be elevated in cancer, and is associated with suppression of the immune system response to tumor cells. As shown in FIG. 33 in an animal model of lung cancer (as described above) expression of STAT3 in primary tumor tissue is reduced in animals receiving radiotherapy (TR), animals treated with a nutritional supplement containing selenium and fish oil (PTN, TN), and animals treated with both radiotherapy and the nutritional supplement (PTRN, TRN). It should be appreciated that treatment with the nutritional supplement at least complements the effects of radiotherapy in regard to tumor STAT3 expression, and can be as effective as radiotherapy.

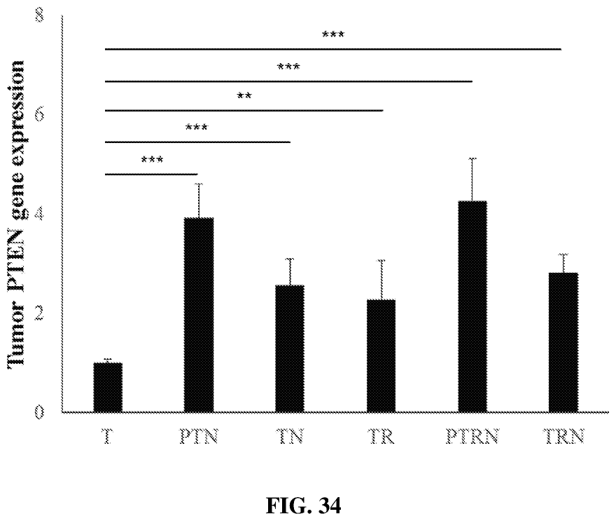

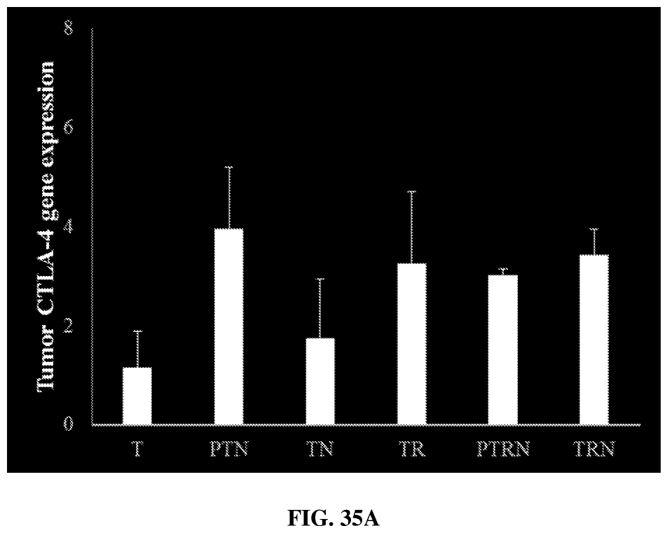

As noted above in studies directed to animal models of breast cancer, PTEN suppression is characteristic of tumor cells. FIG. 34 shows data from a study performed using an animal model of lung cancer, where a nutritional supplement containing selenium and fish oil was used in combination with radiotherapy (as described above). As shown, PTEN expression is low in tumor tissue of untreated animals (T) and is moderately increased in animals treated only with radiation (TR). Treatment with the nutritional supplement was found to increase PTEN expression in the tumor tissue (PTN, TN, PTRN, TRN), particularly when animals were treated with the supplement at the time of implantation (PTN, PTRN).

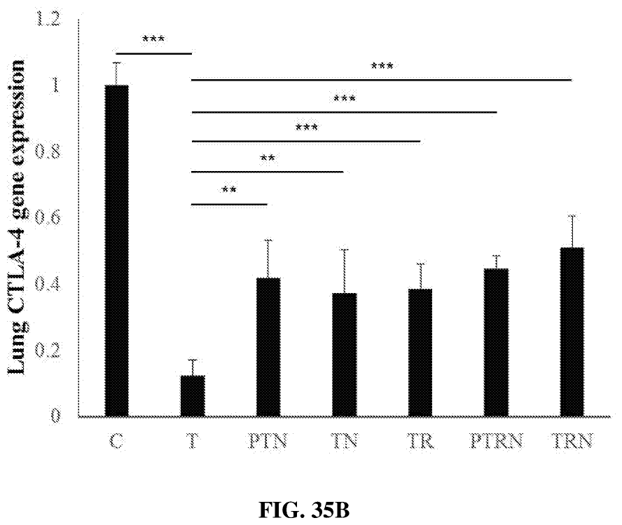

As noted above in studies directed to animal models of breast cancer, CTLA-4 is considered an indicator of T cell activation. Results of studies characterizing CTLA-4 expression in tumor cells obtained from primary tumor cell inoculation sites in an animal model of lung cancer (as described above) are shown in FIG. 35A. As shown, samples from untreated tumors (T) show low levels of CTLA-4 expression, which is increased on radiotherapy (TR). Use of a nutritional supplement containing selenium and fish oil is also associated with increased CTLA-4 expression in tumor tissues (PTN, TN, PTRN, TRN), and is at least complementary to concurrent radiotherapy (PTRN, TRN). Interestingly, treatment with the nutritional supplement at the time of implantation (PTN, PTRN) provides the greatest enhancement of CTLA-4 expression. FIG. 35B shows similar results for studies performed on tissue obtained from the lung (i.e. metastatic sites).

The Applicant believes that the modulation in gene expression levels found in certain tissues following the administration of a nutritional supplement that includes selenium and fish oil (without our without cotherapy using chemotherapeutic drugs and/or irradiation) can be due to changes in expression in tumor cells, changes in expression in surrounding tissue, a result of infiltration of tumor tissue and/or surrounding tissue by cells of the immune system, and/or changes in expression in cells of the immune system. The Applicant notes that many of the changes in gene expression resulting from treatment with a nutritional supplement are consistent with changes seen in activation of cells of the immune system towards anti-tumor activity.

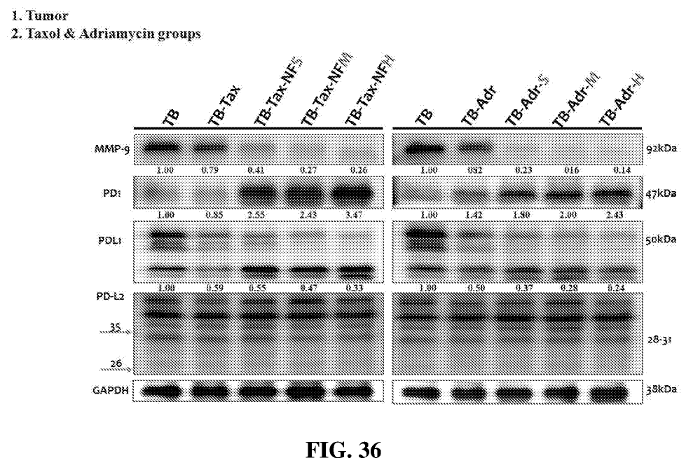

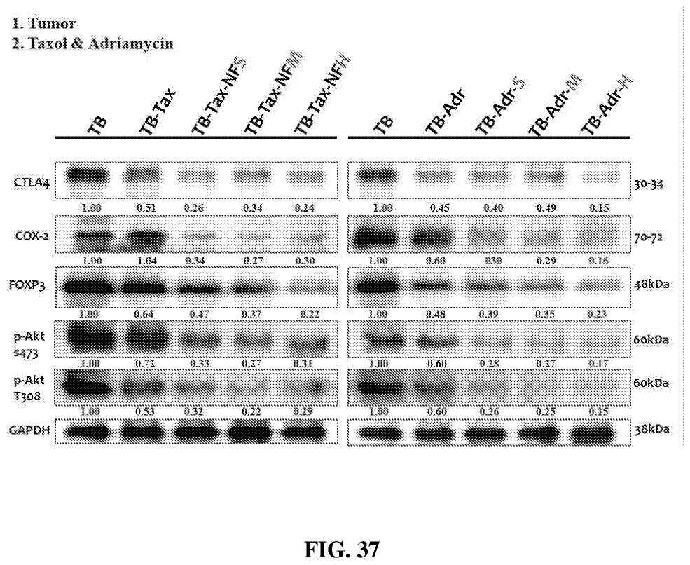

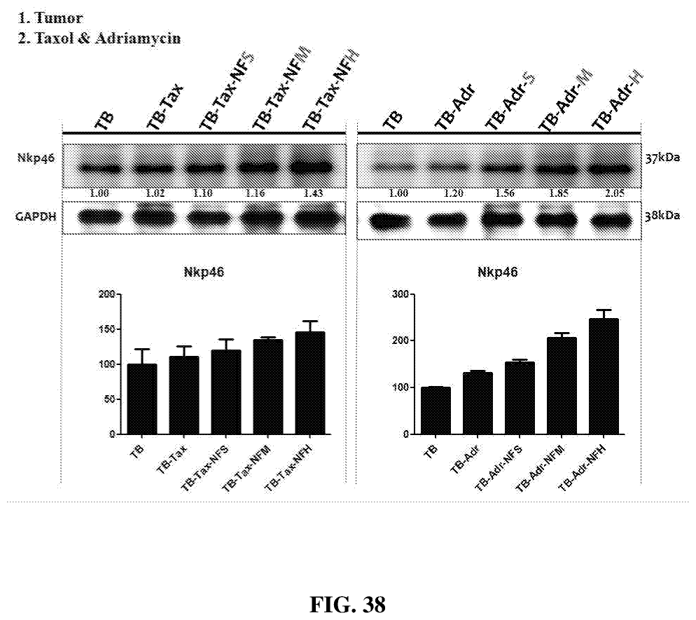

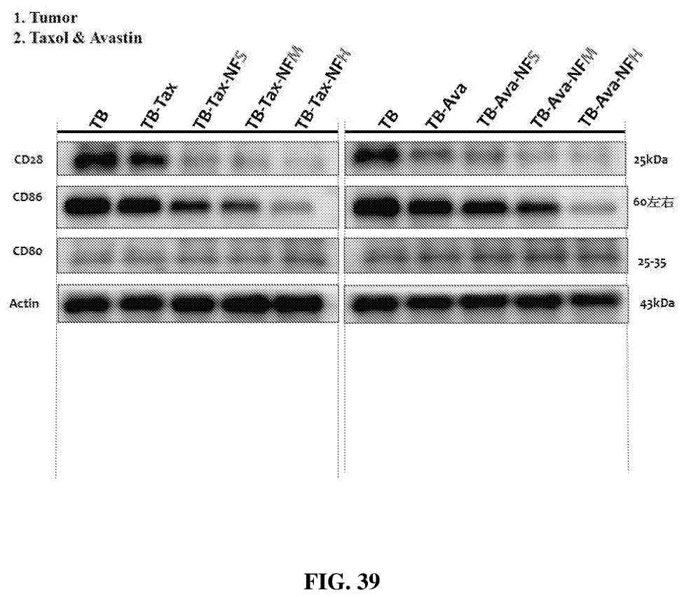

Another embodiment of the inventive concept are methods and compositions for reducing invasiveness and/or angiogenesis in tumors, while modulating immune checkpoint proteins of the tumor. The Inventor has identified selenium and fish oil dose-dependent modulation of proteins associated with tumor angiogenesis and invasion (in addition to immune checkpoint proteins) in an animal model of triple negative breast cancer. Results are shown in FIGS. 36 to 39. FIG. 36 shows dose dependent modulation of MMP-9, PD-L1 and PD-L2 in tumors of animal models of triple negative breast cancer using selenium and fish oil in combination with either Taxol or Adriamycin. FIG. 37 shows dose dependent modulation of CTLA4, COX-2, FOXP3, p-Akt s473, and p-Akt T308 in tumors of animal models of triple negative breast cancer using selenium and fish oil in combination with either Taxol or Adriamycin. FIG. 38 shows dose dependent modulation of Nkp46 in tumors of animal models of triple negative breast cancer using selenium and fish oil in combination with either Taxol or Adriamycin. FIG. 39 shows dose dependent modulation of CD26, CD80, and CD86 in tumors of animal models of triple negative breast cancer using selenium and fish oil in combination with either Taxol or Avastin.

Another embodiment of the inventive concept is a method of treating cancer by treating blood or immune cells (such as NK cells) isolated from patient blood (for example, by leukophoresis) with active components of a nutritional supplement as described above (such as selenium and/or fish oil). Such ex vivo treatment can activate blood or immune cells and/or modulate expression of markers associated with improved antineoplastic immune function. Such ex vivo stimulation can take place prior to, during, or following expansion of the isolated immune cells in tissue culture. Following stimulation the activated/modified immune cells are returned to the patient where they show enhanced antineoplastic activity relative to corresponding non-stimulated immune cells. Optionally, such stimulated cells can be cultured and their numbers expanded (e.g. by clonal expansion) prior to re-implantation. In some embodiments such isolated immune cells can be modified by other methods (e.g. by genetic manipulation) to enhance anti-tumor activity in addition to exposure to active components of a nutritional supplement containing selenium and fish oil.

In some embodiments of the inventive concept ex vivo treatment of immune cells is combined with radiotherapy. In some embodiments radiotherapy can be applied to the individual prior to collection of the immune cells, such that ex vivo stimulation using active components of the nutritional supplement is directed to irradiated cells. In other embodiments ex vivo stimulation of the immune cells takes place prior to radiotherapy, such that irradiation is applied to immune cells that have been pre-treated with active components of the nutritional supplement, or to a patient following infusion of such treated cells. In still other embodiments immune cells collected from a patient can be isolated and treated with active components of a nutritional supplement of the inventive concept while they are subjected to radiotherapy. In a preferred embodiment such radiotherapy is relatively mild compared to those intended to kill tumor cells, and is sufficient to result in a degree of activation, stimulation, and/or otherwise improved anti-neoplastic function in the immune cells without generating common side effects of radiotherapy (e.g. immune suppression, gastrointestinal symptoms, hair loss, mucus membrane damage, skin lesions, etc.).

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

1-52. (canceled)

53. A method providing immunotherapy to an individual with cancer, comprising administering a nutritional supplement comprising selenium and fish oil in an amount sufficient to modify expression of a biological marker associated with an anti-neoplastic immune function.

54. The method of claim 53, wherein the nutritional supplement is provided in the absence of a cotherapy selected from the group consisting of a chemotherapy, a radiotherapy, an antibody-based immunological therapy, and a cell-based immunological therapy.

55. The method of claim 53, wherein the nutritional supplement is provided in an amount sufficient to reduce expression of the biological marker, and wherein the biological marker is selected from the group consisting of AXL, HSP90, p-mTOR, PDL-1, EGFR, HDAC1, p-H2X, p-Akt, pSmad, mTOR, p-PTEN, p-STAT3, CXCR4, STAT3, PD-1, CTLA4, FOXP3, CD8, PTEN, and p-P53.

56. The method of claim 53, wherein the nutritional supplement is provided in an amount sufficient to reduce ratio of expression of CD4 to expression of CD8.

57. The method of claim 53, wherein the nutritional supplement is provided in an amount sufficient to reduce expression of a biological marker associated with a stem cell characteristic or metastatic potential in tumor cells.

58. The method of claim 57, wherein the biological marker of stem cell character or metastatic potential is selected from the group consisting of CD24, CD29, CD31, VEGF, and MMP-9.

59. The method of claim 53, wherein the nutritional supplement is provided in an amount sufficient to increase percentage of CD3+ T cells, CD3+CD4+ T cells, or CD4+CD8+ T cells in the individual's spleen.

60. The method of claim 53, comprising a step of identifying the cancer as drug resistant.

61. A method of activating immune cells to enhance anti-tumor activity, comprising:
isolating an immune cell from an individual to be treated for cancer;
contacting the isolated immune cell with an activating formulation comprising selenium and fish oil in an amount effective to modulate expression of a protein associated with immune cell activation to generate an activated immune cell; and
administering the activated immune cell to the individual.

62. The method of claim 61, comprising a step of clonally expanding the activated immune cell to generate a plurality of activated immune cells, wherein the plurality of activated immune cells is administered to the individual.

63. The method of claim 61, comprising a step of clonally expanding the immune cell to prior to contacting with the activating formulation to generate a plurality of activated immune cells, wherein the plurality of activated immune cells is administered to the individual.

64. The method of claim 61, comprising the step of genetically modifying the immune cell or the activated immune cell.

65. A method of modulating expression of an immune checkpoint or immunotherapy-associated protein in a cell, comprising administering a supplement comprising selenium and fish oil.

66. The method of claim 65, wherein the supplement is administered to provide a concentration of at least 200 ng/mL of selenium at the cell.

67. The method of claim 65, wherein the supplement is administered to provide a concentration of at least 75 μM fish oil at the cell.

68. The method of claim 65, wherein the cell is a cancer cell.

69. The method of claim 68, wherein the cancer cell has stem cell characteristics or is resistant to a chemotherapeutic drug.

70. The method of claim 65, wherein the modulation is a reduction in expression, and wherein the immune checkpoint or immunotherapy-associated protein is selected from the group consisting of PD-L1, p-HSP27, vimentin, p-mTOR, p-p38, β-catenin, ABCG2, CD133, N-cadherin, p-MET, COX-2, GRP78, CD24, CD29, EGFR, HDAC1, p-H2X, p-Akt, MMP-9, CTLA4, CD28, CD86, C31, and STAT3.

71. The method of claim 65, wherein the modulation is an increase in expression, and wherein the immune checkpoint or immunotherapy-associated protein is selected from the group consisting of PD-1, p-AMPKα, E-cadherin, CHOP, FOXP3, Nkp46, CD8, and PTEN.
